(19) United States
(12) Patent Application Publication
(10) Pub. No.: US 2014/0271899 A1
(43) Pub. Date: Sep. 18, 2014

(54) METHOD OF ENHANCING THE BIODISTRIBUTION AND TISSUE TARGETING PROPERTIES OF THERAPEUTIC CECO2 PARTICLES VIA NANO-ENCAPSULATION AND COATING

(71) Applicant: Peroxyium, Inc., Delaware C. Corp., Lebanon, NH (US)

(72) Inventors: James Leiter, Woodstock, VT (US); Susan Gillmor, Vienna, VA (US); Aleksandar Jeremic, Potomac, MD (US); Ekaterina Vert-Wong, Potomac, MD (US); Gregg Fairbrothers, White River Junction, VT (US)

(73) Assignee: PEROXYIUM, INC., DELAWARE C CORP., Lebanon, NH (US)

(21) Appl. No.: 14/213,891

(22) Filed: Mar. 14, 2014

Related U.S. Application Data
(60) Provisional application No. 61/802,915, filed on Mar. 18, 2013.
(60) Provisional application No. 61/785,794, filed on Mar. 14, 2013.

(51) Int. Cl. A61K 9/51; A61K 33/24

(52) U.S. Cl. CPC A61K 9/5123; A61K 33/24
USPC 424/498; 424/502; 424/617

(57) ABSTRACT

The present invention provides methods and liposomal compositions useful in therapeutics, and diagnosis, prognosis, testing, screening, treatment and/or prevention of various disease conditions. The present invention provides imaging methods for various conditions. The present invention is a multi-layered drug delivery pathway, inclusive of nanoparticle liposomal formulations and mechanisms of localized action via unzipping upon delivery to the affected tissue site. The nano-encapsulation methodology allows maximization of a potent antioxidant's biocompatibility, increased target cell penetration and uptake, reduced off-target effects and retention of high anti-oxidative activity for promising therapeutic potential.

### CROSS-REFERENCE TO RELATED APPLICATIONS

This application claims priority to of U.S. Provisional Application No. 61/785,794 filed Mar. 14, 2013 and U.S. Provisional Application No. 61/802,915 filed Mar. 18, 2013, the contents of each is incorporated by reference herein in their entirety.

### FIELD OF THE INVENTION

This invention relates to field of nanotechnology, pharmacology, medicinal chemistry and engineered liposomes invented to enhance the properties of previously tested compounds that are available in the public domain.

### DESCRIPTION OF THE BACKGROUND

In developed countries chronic diseases, so-called diseases of civilization, comprise the bulk of morbidity, mortality, and challenges to quality of life, as well as the biggest drivers of cost in healthcare. Inflammation by reactive oxygen and nitrogen radicals is intimately implicated in these diseases, including obesity and diabetes, pulmonary diseases, neurodegenerative disorders, stroke, atherosclerosis, myocardial infarction, chronic heart failure, circulatory shock, arthritis and chronic auto-inflammatory diseases.

Reactive Oxygen Species (ROS—primarily superoxide, O₂⁻, and its derivatives) and Reactive Nitrogen Species (RNS—primarily nitric oxide, NO, and its derivatives) are major contributors to inflammatory damage in biological organisms. ROS- and RNS-generating enzymes are found in virtually all human tissues. Inflammation leads to the generation of these Reactive Species (RS) and oxidative stress. The oxidation, nitration and hydroxylation activities of these RS are thought to be key mechanisms in aging and in a wide range of age-associated chronic diseases disclosed herein. Most are progressive, and all have in common strong evidence of pathogenic inflammation and oxidative-nitrative cellular injury and death. There is strong evidence that the activity of RS is central to cells' life and death decisions in homeostasis or the initiation of apoptosis and necrosis.

In particular among the RS, peroxynitrite (ONOO⁻) is formed by a diffusion-controlled reaction of O₂⁻ and NO in the fastest in vivo reaction known to biology. It is an extremely powerful oxidizing and nitrating agent, and unlike the highly toxic hydroxyl radical, peroxynitrite has a half-life long enough to diffuse among different cells and propagate oxidative organ damage. It causes extensive and often cytotoxic oxidative and nitrative damage to proteins, lipids, DNA, RNA, and carbohydrates and in addition, triggers chronic feedback loops that can overwhelm the body's antioxidant defenses. Over long periods of time this oxidative cascade can outlive the original inflammatory insult and create an indolent and persistent, self-sustaining inflammatory state (as discussed further herein). As a strong oxidizing and nitrating agent, peroxynitrite targets key cellular components causing tissue injury. Peroxynitrite is implicated in many pathophysiologic conditions, and the body's own systems are ill-equipped to eliminate it. Agents that directly interfere with peroxynitrite activity have been suggested as therapeutic tools in combating inflammatory chronic diseases.

Free radicals are formed as a result of mitochondrial dysfunction, which accompanies a large number of central nervous system (CNS) disorders, and the actions of heme-oxygenase, myeloperoxidase, xanthine oxidase and NADPH oxidase, which may generate free radicals in a variety of inflammatory conditions. The free radicals responsible for tissue damage include the superoxide radical, nitric oxide and peroxynitrite (formed from the superoxide radical and nitric oxide, which is formed by and nitric oxide synthases—endothelial, neuronal, and inducible). Peroxynitrite is probably the most damaging of these free radicals due to its relatively long half-life and high reactivity (1). Evidence of oxidative damage is detected by the residue it leaves behind: peroxidation of lipids and nitration of proteins, especially tyrosine. Evidence of lipid peroxidation and nitration of proteins is wide spread in multiple sclerosis (MS), a variety of degenerative brain diseases (amyotrophic lateral sclerosis (ALS), Parkinson's disease, Alzheimer's disease, Traumatic Brain Injury, etc.), ischemic brain damage, traumatic brain injury and in systemic disease such as heart failure, Chronic Obstructive Pulmonary Disease (COPD) and diabetes (2-11).

Lung Diseases

Many lung diseases, including chronic obstructive pulmonary disease (COPD), asthma, bronchiectasis, cystic fibrosis, and interstitial lung disease, involve chronic inflammation and oxidative stress. COPD is the fourth leading cause of death in the US, accounting for approximately 4.5% of all deaths per year. Prevalence estimates range to 13,500,000 plus “undiagnosed” up to 15,000,000; 2 million have emphysema. It is a heterogeneous disease caused by inflammation, edema, and secretions, which result in morphological changes in all regions of the lungs. Lung function declines with age, manifesting as progressive, irreversible organ failure notably in emphysema and chronic bronchitis. These disorders represent major burdens of disability and mortality world-wide, and currently no therapies short of whole lung transplant significantly change their natural history.

Enhanced inflammation in the lungs is a prominent characteristic feature in emphysema/COPD, asthma, and other degenerative lung diseases such as idiopathic pulmonary fibrosis. Characteristic of these diseases, oxidative stress is critical to inflammatory responses and pathogenic mechanisms in the chronic inflammation, remodeling of extracellular matrix and blood vessels, elevated mucus secretion, inactivation of anti-proteases, apoptosis, autophagy and regulation of cell proliferation. Established evidence of RS-mediated cellular damage is substantial and includes carbonyl-modified or tyrosine-nitrosylated proteins, which impair protein and enzyme function; lipid peroxidation, which damages cell and organelle membranes; changes in levels of hydrogen peroxide (H₂O₂) and nitric oxide (NO); increased levels of pro-inflammatory cytokines and decreased levels of glutathione, a principal physiological antioxidant in the lung; inactivation of anti-proteases and activation of matrix metallo-proteinases (MMPs) causing an imbalance of proteases/anti-proteases, which leads directly to cellular injury and death; DNA and RNA oxidation in alveolar wall cells, which causes programmed cell death; and breakdown of extracellular matrix through increased release of elastolytic enzymes, which promotes tissue degradation characteristic of emphysema.

Diseases of the Central Nervous System (CNS)

As a result of the high levels of oxygen required, the brain is particularly sensitive to ROS-mediated damage. Behind the blood-brain barrier, it has long been suspected that oxidative stress generated by leakage from normal mitochondrial respiration and respiratory bursts of RS from activated microglia contribute to neuronal death in intractable diseases of the central nervous system, including Alzheimer's Disease, Parkinson's Disease, Traumatic Brain Injury, Multiple Sclerosis, Senile Dementia, Amyotrophic Lateral Sclerosis and others as discussed herein. Studies have found evidence of oxidative damage to DNA, lipids, proteins, calcium balance, and neurotransmitter activity in what can become a vicious and self-perpetuating, autotoxic cycle, especially in brains of elderly subjects. Markers of RS activity have been found in all the major CNS diseases. The most reliable risk factor for neurodegenerative diseases is aging, suggesting that during senescence, the brain may become more vulnerable to RS insults and/or that their effects may be compounded over long periods of time. “Most, if not all, models of cell death involve free radical species and oxidative stress. It may thus be possible to interfere with cell death in the neurodegenerative diseases by devising therapeutic strategies aimed at stopping or slowing free radical-mediates oxidative damage.” (12)

Parkinson's disease (PD), the second most common neurodegenerative disease of adults, is usually a sporadic, non-hereditary condition involving loss of dopaminergic neurons from the substantia nigra pars compacta and the presence of prominent eosinophilic intracytoplasmic proteinaceous inclusions termed Lewy bodies and neuritis. PD is characterized by resting tremor, bradykinesia (slowed ability to start and continue movements, and impaired ability to adjust the body's position), rigidity, and postural instability. The disease is chronic and progressive. Patients experience increasing difficulty in daily living functions as the disease progresses. PD affects approximately 1% of the population by age 65 years, increasing to 4% to 5% by the age of 85. Prevalence is approximately 1,000,000 in North America, with an annual incidence of 50,000. While levodopa has improved quality of life for PD patients, population-based surveys suggest these patients still display decreased longevity compared to the general population. Furthermore, most PD patients suffer considerable motor disability after 5-10 years of disease even when expertly treated with optimum medical therapy, and there is accumulating evidence that L-dopa-enhanced dopamine oxidation accelerates loss of dopaminergic neurons.

Open angle glaucoma (OAG) is associated with ocular hypertension and progressive loss of vision, in many cases despite adequate control of intraocular pressure. Glaucoma is the second leading cause of blindness world-wide (13). Blindness occurs as retinal ganglion neurons (RGNs) are killed, and the processes that kill RGNs may extend into the central nervous system to additional neurons in the visual pathways (14). Hence, progression of OAG is actually a neurological disease. The mainstay of treatment for OAG is medical therapy to facilitate the removal of intraocular fluid through the canal of Schlemm, through which intraocular fluid is drained, or suppress the formation of ocular fluid, all with the aim of decreasing intraocular pressure (13). If medical therapy fails, a variety of surgical procedures have been developed to improve drainage of ocular fluid from the eye. Despite these, therapies, many patients continue to lose visual acuity. Free radical formation either as a response to elevated intraocular pressure or as a process, perhaps related to aging, independent of intraocular pressure plays a prominent role in the loss of visual function in OAG (15, 16). Antioxidant therapies have been beneficial in animal models of OAG (14, 16-21), though no neuroprotective, antioxidant therapy is currently approved for use in glaucoma.

Cardiovascular diseases are a leading cause of mortality and morbidity worldwide, and hypertension is a major risk factor for cardiovascular disease and stroke. Numerous studies support the contribution of reactive oxygen and nitrogen species in the pathogenesis of hypertension, as well as other pathologies associated with ischemia/reperfusion. These diseases affect more than 600 million people, and it is estimated that 29% of the world's adult population will suffer from hypertension by 2025. The pathophysiology of cardiovascular diseases is complex due to the multiple biological pathways that have been implicated, but these diseases often originate in the vascular endothelium. Following endothelial activation, oxidative stress has an important role in the development of atherosclerosis and hypertension, thereby contributing to the progression of the structural and functional cardiovascular damage. In cardiovascular disease related to ischemia/reperfusion injury, redox imbalance triggers the activity of a number of signaling pathways mediated by ROS and RNS. Consequently, in cardiac surgery with extracorporeal circulation, electrical and structural myocardial remodeling due to the excessive production of these reactive species may lead to the development of arrhythmias such as atrial fibrillation. Furthermore, reperfusion injury after acute myocardial infarction results from increased ROS and RNS formation, and the oxidative stress of reperfusion may enhance the infarct size. These cardiac abnormalities are associated with major changes in oxidative stress-related biomarker. Antioxidant therapy should be effective in the early stages of hypertension or atherosclerosis by preventing the oxidative-stress mediated “positive feedback loop” of progression from reversible endothelial dysfunction to atherosclerotic plaque formation.

Despite abundant evidence of oxidative damage to DNA, proteins and lipids, therapeutic trials with antioxidants have been almost universally disappointing. The efficacy of antioxidant therapies is contingent upon several factors (22). First, the therapeutic reagent must localize to affected tissues (for example, cross the blood brain barrier). Second, the compound must accumulate in the affected tissues at a high enough concentration to be clinically effective in the treatment of the disease. In case of the CNS diseases, fewer than 2% of ‘small molecule’ drugs are capable of penetrating the blood brain barrier, and only a fraction of these have appreciable deposition in the brain (23, 24). In other systemic diseases, drug penetration and maintenance of adequate drug levels over the duration of treatment also limit the effectiveness of antioxidant therapies. Last, the therapeutic agent must have a long half-life sufficient to neutralize excessive amounts of Reactive Oxygen Species (ROS) produced as part of chronic disease process. Most antioxidants fail one or more of these requirements for effectiveness.

The inventors tested potent synthetic, antioxidant cerium oxide (“ceria” or CeO₂) nanoparticles (CeNPs) capable of neutralizing the superoxide anion, hydrogen peroxide, nitric oxide and peroxynitrite in an in vitro model of stroke (26). The chemical reactivity of these particles is regenerative as the CeO₂ cycles between the +4 and +3 valence states (26-29). In addition, the small size, biocompatibility and charge of the CeNPs results in wider biodistribution and more effective central nervous system penetration than other formulations of nanoparticles (26, 30-33). It is believed that differences between the physical and chemical properties of the particles among different studies determine how the particles react with various biological interfaces and may underlie the dramatic differences in the distribution and biological effects of these materials (34-36).

Most of the therapeutic potential of ceria has been assessed using in vitro or cell culture models (37-39) or in vivo in models with no clear clinical correlate (40). Recently, we demonstrated the effectiveness of CeNPs in an animal model of Multiple Sclerosis using clinically relevant, behavioral endpoints (33). In this study, CeNPs were as effective as fingolimod, an FDA-approved drug for use in Multiple Sclerosis in humans. Moreover, CeNPs have reduced retinal damage (41), reduced the size of infarcts in a middle cerebral artery model of ischemia in rodents (42) and improved cardiac function in a murine model of cardiomyopathy (43). In all of these studies, the beneficial effects of CeO₂ nanoparticles have been attributed to the antioxidant activity of the particles.

### SUMMARY OF THE INVENTION

The present invention is based, in part, on the discovery that multi-layered encapsulation of cerium oxide particles is useful for enhancing their anti-oxidative activity, maximization of potent antioxidant's biocompatibility, increase in particles' target cell penetration and uptake, reduction of off-target effects and retention of high anti-oxidative activity. Accordingly the present invention provides methods and liposomal compositions useful for a variety of entities, especially therapeutic entities, and that are useful in the diagnosis, prognosis, testing, screening, treatment or prevention of a disease condition. In one embodiment, the methodologies and compositions of the present invention are useful for directing the reaction between cerium oxide nanoparticles and reactive oxygen species.

The present invention provides imaging methods for various conditions as described herein. Imaging using the cerium oxide nanoparticles use the intrinsic fluoresce properties of Ce⁺³ and Ce⁺⁴, direct chemical attachment of commercial dyes to the particle surface and incorporation of dyes via the encapsulated lipid layer. In another embodiment, the present invention provides a multi-layered drug delivery pathway, inclusive of nanoparticle liposomal formulations and mechanisms of localized action via unzipping upon delivery of the formulation/composition to an affected tissue site as described herein. In yet another embodiment, the nanoparticle liposomal formulations also have a multifunctional hydrocarbon interface between the liposomal encapsulation and have a radical stability to shuttle electrons to and from the cerium oxide nanoparticles. These developments of nano-encapsulation method maximizes the antioxidant's biocompatibility, increases target cell penetration and uptake, reduces off-target effects and enhances retention of high anti-oxidative activity for therapeutic potential.

In another embodiment, the present invention provides methods to control and direct the desired CeNP action against reactive oxygen species via shedding of the biocompatible layer encapsulating it for near contact (unzipping route) and/or via extended electronic sphere of CeNP radical interaction using stable radical surface moieties derived from a hydrocarbon linker interposed between the CeNP surface and the lipid encapsulation. The encapsulation of CeNPs prevents the interaction of the CeNP with biological materials in blood and tissues where free radical concentrations are not elevated. The encapsulation is ‘unzipped’ by the presence of free radicals so that the anti-oxidant activity of CeNPs is made available most readily at sites with the body where free radicals are formed or are abundant. The unzipping is achieved in two embodiments. In the first embodiment, the lipids encapsulating the CeNP are linked to the surface of the citrate treated, for example, surface of the CeNP using specific chemical bonds. In the second embodiment, short linking hydrocarbons are interposed between the lipid coat and the citrate treated CeNP surface. The chemical bonds linking the lipids or hydrocarbons to the citrate treated CeNP surface are more or less susceptible to chemical attack by free radicals, and the chemical bond linking the lipid encapsulation to the hydrocarbon linker is also more or less susceptible to attack by free radicals, such as superoxide and peroxynitrate. Moreover, the hydrocarbon linkers may possess chemical structures to enable electron shuttling to the CeNP surface, promoting a larger range of free radical scavenging the distal moieties (distal from the CeNP surface) of the hydrocarbon linker, which form stable free radicals themselves. This creates a double unzipping process when hydrocarbon linkers are present and extends the range of antioxidant activity from the CeNP core. The susceptibility of the double unzipping bonds at each end of the hydrocarbon linker need not be similar. For example, one might have the lipid to hydrocarbon bond be very susceptible to free radical attack and the inner, hydrocarbon to CeNP bond be less susceptible to free radical attack. Many permutations with variable free radical attack bond susceptibilities are possible. Thus, by controlling the range of radical interactions with CeNP (from short to long depending on the length of the hydrocarbon linker), the present invention provides a variety of formulations that encompass applications of the described compositions/formulations for long term dosage in a variety of chronic inflammation diseases, with a low toxicity profile and maximized therapeutic or diagnostic potency. The present invention provides formulations that bring CeNP and radicals together for action both through near contact and extended contact ranges.

In yet another embodiment, the present invention is based in part on a multi-layered encapsulation of cerium oxide particles that is useful to create an “off-switch” to the intrinsic anti-oxidative activity of the CeNPs, and the layered encapsulation limits the interaction of the encapsulated CeNPs to interact with blood and tissue while the encapsulated CeNPs circulate in the body. Limiting the anti-oxidant activity during administration and transit of the encapsulated CeNPs to the sites of inflammation enhances biocompatibility. Such encapsulation allows complete or partial reduction of off-target effects. In one embodiment, this is based, in part, on coating CeNP in a specific way so that the CeNPs are not active. The CeNP redox activity is suppressed by a coating, such as a lipid and hydrocarbon coat. This novel strategy prevents pro-oxidant effects while the passivated CeNP is introduced into living tissue. In one embodiment, this provides for a research and diagnostic tool, as well as a strategy to emphasize safety of a therapeutic formulation, thus enabling control of the ratio of safety-to-efficacy in therapeutic settings. In another embodiment of the present invention, the method of passivating the CeNP anti-oxidant activity reduces off-target uptake and off-target effects by suppressing the anti-oxidant activity of CeNP at those biological sites that lack significant free radical formation, which is necessary to unzip the encapsulated, passivated CeNPs.

Accordingly, the present invention provides methodology for passivating a CeNP by limiting its reactivity. The invention allows for more or less coverage, long hydrocarbons, and bulkier side chains (e.g., tert-butyl group(s)) in the riddle of the hydrocarbon chain, and other functional groups that block or interfere with CeNP chemical activity. In another embodiment, this novel formulation approach is important as a research tool in optimizing the manufacturing process for these particles when used as therapeutics and/or diagnostics, as well as improving the ratio of therapeutic effect and/or organ toxicity.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 demonstrates the first phase of creating the multi-layered drug delivery pathway for cerium oxide nanoparticles by preparing them as liposomally encapsulated particles. Building of a ligand shell on top of the CeNP particles for surface stabilization allows adding specific tissue targeting capability into liposomal formulations. (A) Schematic of the surface reactive groups on CeNPs shows the available carboxylic acid groups. (B) Direct surface modification (citric acid as hydrophilic molecule is shown in this example) prepares a surface of the CeNP for the attachment of targeting molecules by chelating the CeNP. While citric acid ligand is given as an example here, there are over 1800 carboxylic acid compounds, which give rise to numerous permutations of the particle surface chemistry.

FIG. 2 demonstrates an example of how a ligand shell around CeNP maximizes the antioxidant's biocompatibility. This example shows the ligand shell surrounding the CeNP particles with oleic acid. (A) By attaching a lipid that may vary between 8 to 20 carbons in length as a ligand to the CeNP surface, the terminal carboxylic acid of the lipid complexes with the surface while the hydrocarbon tail creates a hydrophobic surface around the CeNP core. (B) Illustrating the overall presentation of oleic acid surface of CeNP. (C) Structure of oleic acid. The carboxylic acid binding properties of citric acid and its congeners are key to build the ligand shell. The choice of inert hydrocarbon (butyl, t-butyl, hexyl, decyl, hexyldecyl, etc.) or reactive end-groups in the initial interaction of the CeNP with the surface treatment (citric acid in this figure) depends on the desired functional outcome. Carboxylic acid ligands with reactive or protected groups (azides, alkynes, thiols, protected amines, protected carboxylic acids etc.) allow for maximum possible modifications and surface chemistry flexibility directly at the surface of the CeNPs.

FIG. 3 illustrates a composition of tailored formulation including CeNP particle coated with lipid/PEG hybrid layer. While DOPC and PEG350 PE are shown, a variety of lipids may be introduced to tailor the outer surface of the CeNP encapsulation for specific applications. Phospholipids are a major lipid component in cell membranes and the choline head group does not participate in cell signally, making it a logical, inert choice for lipid encapsulation to enhance biocompatibility. However, the example should not be seen as a limit on the possibilities of encapsulating the nanoparticles with a wide variety of lipids for possible tissue targeting.

FIG. 4 illustrates functionalization of the hybrid layer. To attach targeting molecules, lipids are incorporated with reactive head groups into the lipid hybrid layer, showing two of several possibilities. This strategy would improve target cell penetration and increase selective cellular uptake of cerium oxide nanoparticles.

FIG. 5 illustrates one example from our methodology of synthesizing of an unzipping particle. We demonstrated the steps of the methodology of synthesizing cerium oxide nanoparticles that unzip and shed the lipids bound to the surface of the particle upon encountering ROS and/or RNS. In this example, after functionalizing a ligand layer to a terminal thiol (—SH) group, CeNPs are exposed to thiol lipids or alkane thiols (hydrocarbons with terminal thiols) to form a di-sulfide bond. When a particle is subsequently exposed to lipids, such as DOPC or others tailored to the specific CeNP application (such as PEG modified lipids), a bilayer results. The logic of the selection of the preferred linkage is not part of this figure, which only demonstrates the principle of building the chemical attachments.

FIG. 6 illustrates how the action of unmasking of the active ingredient via an unzipping process is created by the chemistry of the surface modifications and the specific chemical bonds used to attach lipids or short hydrocarbon linkers to the CeNP or between the lipid outer surface and a short hydrocarbon linker, which is bound to the CeNP surface. When CeNP unzipping particles are exposed to DTT (dithiothreitol) in vitro or glutathione in vivo, the di-sulfide bonds will be cleaved to regenerate the ligand surface. After shedding a protective lipid/PEG layer, the CeNP is ready to act as an antioxidant agent in the cellular environment.

FIG. 7 illustrates (A) the route to various permutations and attachment strategies of ligand shell modification. This example, which uses citric acid as the initial treatment of the CeNP surface, demonstrates that other amine coupling reactions are possible using the available carboxylic acid on the citric acid ligand. There are 5000 possible amines for coupling available from a single market source, such as Aldrich catalogue. Hence, there is great flexibility in tuning the lipid to CeNP bond by varying the initial CeNP surface treatment to make the connection between the CeNP surface and the outer encapsulation more or less susceptible to attack, and unzipping of the core CeNP, by free radicals. (B) Illustrates direct attachment of dopamine using a citric acid ligand. (C) Illustrates attachment of L-DOPA with BMPH as a spacer for increased accessibility to the dopamine receptors using the citric acid ligand. The thiol terminated surface offers the direct attachment of other thiol terminated small molecules or cysteine terminated peptides. (D) Illustrates direct attachment of L-DOPA using an amine terminated CeNP surface. L-DOPA is used for concept illustration in this drawing.

FIG. 8 illustrates attachment of (a) L-DOPA and (b) a generic peptide to a thiol lipid head group on a lipid/hybrid bilayer. Using the same strategy, peptides with a free cysteine are easily added to the lipid layer for further particle tailoring. A primary amine in the lipid head group gives rise to alternative potential modification of the lipid layer.

FIG. 9 shows two different attachment strategies to modify the CeNP with fluorescent dyes for therapeutic, diagnostic and research applications. Dyes may also be introduced via lipids (FIGS. 3-6). These dyes, coupled with the intrinsic fluorescent properties of Ce⁺³, enable tracking of the particle, its shell, their interactions together and their interactions in cells, tissues and animals.

FIG. 10 lists the enthalpies (ΔH°) to form free radicals of various chemical functional groups. Coupling the bond dissociation energy (ΔH° (BDE)) and the bond formation (ΔH° (BFE)) energies enable an estimation of the energetic cycle (44) (45-47). In the presence of free radicals, these chemical linkers form stable radicals, and the bond dissociation and formation energies show that bond cleavage and reforming are thermodynamically favored. From this analysis, numerous possible unzipping examples are identified. The susceptibility of the lipid-CeNP layer to free radical unzipping can, thereby, be tailored to the rate of free radical formation and/or the CeNPs can be controlled and released or made available in proportion to the severity of free radical inflammation in any particular tissue.

FIG. 11 shows a schematic diagram of the CeNP coupling to an amine terminated hydrocarbon. Using EDC and NHS, the carboxylic acid of the polyacrylate ligand on the CeNP surface becomes reactive and readily forms an amide bond with the addition of the desired amine. The examples listed, decyl amine and acetal amine, are two successful modification that have been completed.

FIG. 12 tracks the amide coupling reaction on CeNPs via infrared (IR) spectroscopy. (A) Showing IR spectrum of the CeNP with the polyacrylate ligand shell (‘bare’ or stock CeNP) and the spectrum of CeNP-decyl amine (crude extract). (B) Demonstrates decyl amine C—H stretching, N—H stretching and a series of other stretches and wags, and compares CeNP-decyl amide (crude extract). (C) Shows effects of adding citrate to the crude extract on unreacted amines.

FIG. 13 shows the NMR spectra of the unreacted decyl amine compared to the CeNP product. After the reaction, the α-methylene protons (those adjacent to the amide bond) shift from 2.68 to 2.21 ppm. A weak peak at 7.85 ppm appears, which is attributed to the amide proton peak.

FIG. 14 compares the DLS scans of the CeNP starting material with the polyacrylate ligand only (‘bare’) and lipid encapsulated CeNPs. (A) The bare CeNPs have a single particle distribution peak (99% of mass) centered at 0.77±1.0 nm. (B) The lipid encapsulated CeNPs exhibit four peaks. About one third of the population has formed the vesicles in the desired size range (4-8 nm). This population includes only those vesicles that contain a single nanoparticle core. It is also likely that larger liposomes that have formed include the modified nanoparticles as well.

FIG. 15 shows the change in fluorescence when CeNPs are in close proximity to a lipid dye. The intrinsic fluorescence of Ce⁺³/Ce⁺⁴ exhibits an excitation peak at 350 nm and an emission peak at 465 nm, as shown in the unmodified CeNP spectrum. If an appropriate lipid dye is within 10 nm, the fluorescence will shift to the lipid dye. The example lipid dye in this figure shifts the emission peak to 520 nm, indicating that the lipids are within 10 nm of the CeNP.

FIGS. 16(A & B) shows the complex formation of Fe⁺² and 1,10-phenantholine and its absorbance spectrum.

FIG. 17 uses hydrogen peroxide (H₂O₂) decomposition in the presence of Fe⁺²/Fe⁺³ and 1,10-phenantholine (PA) to measure Fe⁺²/Fe⁺³ cycling in the presence of CeNP. The conversion of Fe⁺² to Fe⁺³ indicates high activity and leads to a low number Fe⁺²+PA complexes, which absorb at 520 nm. (A) When comparing all assays, the unmodified CeNPs have the highest activity, although they are the least biocompatible. (B) Without H₂O₂, the maximum absorbance occurs when no H₂O₂ is present. In the assays with CeNP (C), there is a noticeable increase in Fe⁺³, indicating CeNP activity. When comparing the two modified CeNP (D), the acetal, or unzipping nanoparticle, exhibits a higher activity than the decyl amide CeNP.

FIG. 18 illustrates the linear relationship between Fe⁺² concentration when compared to the absorbance of the Fe⁺²+PA complexes at 520 nm. From this calibration curve, the activity assay data can be quantified.

FIG. 19 shows the quantification of the activity assay. Initially, 299 μM of Fe⁺² is in solution. From the calibration curve, the concentration of Fe⁺² is calculated at the inflection point. The maximum absorbance from the assay without H₂O₂ indicates that 219 μM of complex forms out of an initial 299 μM Fe⁺² present. When H₂O₂ is added to the Fe⁺² solution, approximately 88 μM of Fe⁺³ is converted. In contrast, the decyl CeNP increases the amount of Fe⁺³ to 126 μM and acetal CeNP converts 150 μM. ^{§}The unmodified CeNP produces the highest amount of Fe⁺³ in this assay of ˜200 μM. ^{§}All inflection point data are estimated to be the absorbance value at 15 s. *Data from the Fe⁺²/PA control run is used to estimate the maximum absorbance and the maximum free Fe⁺² available. Those absorbance values are takes at t=∞.

### DETAILED DESCRIPTION

The invention summarized above may be better understood by referring to the following description. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

In one embodiment, the present invention enhances tissue targeting and activation of a durable, regenerative catalytic agent that reduces ROS levels, especially peroxynitrite (ONOO⁻)—the most potent and persistent antioxidant in the human body—and delivers the agent to the sites of excess free radical formation within the body. Increased diseased tissues deposition of CeNPs (and decreased liver and spleen deposition) is achieved by incorporating the CeNPs into liposomes and functionalizing the surface of the liposome. Selective unmasking of redox activity is achieved by liposomal coating of the CeNPs that limits activity of the CeNP until the lipid coat is removed by free radical attack. Thus, this embodiment consists of a two-stage process of functional targeting and drug release to enhance tissue-specific redox activity at sites of greatest free radical formation. This embodiment also consists of two strategies: unmasking by complete cleavage and unmasking by the shuttling of the electrons through specific chemical groups on hydrocarbon linkers between the lipid surface and the CeNP surface.

In another embodiment, these engineered nanoparticles shall be used as therapeutic agents for diagnosis, prevention and treatment of chronic diseases such as: systemic illnesses such as COPD-emphysema, asthma, Idiopathic fibrosing pancreatitis (IFP); systemic autoimmune disease such as type-1 diabetes, arthritis and degenerative amyloid-induced brain and pancreatic diseases such as Alzheimer's, Parkinson's, Glaucoma, Macular Degeneration, Traumatic Brain Injury, Cardiovascular diseases and type-2 diabetes mellitus, in which oxidative stress and/or amyloid formation play a pathological role (38, 48-50).

In one embodiment, the present invention provides targeted and tailored surface chemistries for the cerium oxide nanoparticles (CeNPs) to maximize radical scavenger behavior in vivo. The combination of lipid and surface chemistry is critical to balance biocompatibility, surface modification and efficacy. The surface modification is organized into two layers. The first, proximate to the surface, preserves the redox CeNP activity by tailoring coverage and unzipping. The second, building on the first, forms an interface with the first layer to encapsulate the CeNP with lipids and/or polyethylene glycol (PEG) and/or specific proteins to maximize biocompatibility and optimize the circulation time and the specificity of tissue delivery and uptake. Modified CeNPs are described herein and as is the optimization of this strategy. As described herein, the present invention details the unzipping modification and tailoring of the lipid and PEG layers.

The following provides for aspects of the interfaces and the pathways for nanoparticle modification. First, the CeNP surface is chelated with a ligand to enhance stability. The most successful ligands to date are carboxylic acid chelating small molecules. These enhance stability and give additional control over particle size (51-53). Sigma Aldrich has over 1800 carboxylic acid compounds in its catalogue. The present invention is exemplified using citric acid or polyacrylate. However, potential surface modification strategies can be expanded using alternative chelators to create new surface chemical options. For example, butyl, t-butyl, hexyl, decyl, hexyldecyl with n-terminal carboxylic acids will create an inert hydrophobic surface ready for immediate lipid encapsulation. Carboxylic acids with additional functionalities at the terminal position, such as ethers, esters, epoxide, peroxides, thiols, acetals functionalities, embed unzipping capabilities.

In the case of cerium oxide, carboxylic acid chelators, such as citric acid and EDTA (Ethylene diamine tetraacetic acid), EGTA and their derivatives (115 compounds were identified) effectively bind to the surface and lead to the surface stabilization that is required for further modifications to add specific tissue targeting capability into the invention's liposomal formulations. Carboxylic acid compounds with non-reactive terminal groups (for example stearic or oleic acid) create the desired effect via non-specific modification such as lipid hybrid bilayer. Citric acid or polyacrylate and their potential for further modification are explored in the formulations of the invention. Both have multiple carboxylic acid (—COOH) groups, of which at least one is available to attach.

The exposed carboxylic acid group enables the use of carbodiimide and succinimide chemistry (EDC/NHS) to couple the carboxylic acids to amine (—NH2) terminated small molecules such as hydrocarbon amines (butyl, t-butyl, hexyl, decyl, hexyldecyl amine), peptides, functional amines (ethers, esters, epoxide, peroxides, thiols, acetals etc.), L-DOPA, dopamine derivatives and more. This strategy mimics the peptide bond formation and is widely used to couple carboxylic acid moieties to amines (54-56). Sulfhydryl groups can also be taken advantage of here. In addition, Sigma Aldrich offers ˜5000 amines in its catalogue to attach to the exposed carboxylic acid; there are opportunities for great variety and great specificity and control using specific amines in different settings. Outlined herein are a representative number of such amines. Using well-characterized reactive groups, there is a wealth of surface modifications.

Thus far, the current ligand shell (citric acid/poly acrylate) has proven useful for further CeNP surface modification via amine coupling to form an amide bond. It provides for further surface modification. In addition, the amide bond will form a free radical and may act as a shuttle between CeNP and the outer layer, allowing CeNP to be available for conversion of free radicals into less reactive species. It may or may not cleave in the presence of ROS.

Thus far, the ligand shell offers an initial tailoring opportunity. The choice of at least one hydrocarbon addition via amide bond or other chemical coupling (or thiol, azide, alkyne) adds another point of modification.

Another tailoring opportunity (hydrocarbon addition) to chemically modify the CeNP surface is with a 2-40, 4-20, 6-12 or 8-10 carbons hydrocarbon. The hydrocarbon plays two roles: (1) to tune activity and (2) to prepare the CeNP surface for lipid coating. For activity tuning (1), the length of the hydrocarbon can play a role. Long carbon chains (16 to 40 carbons) can completely passivate the surface, while short ones (4 to 12 carbons) may reduce activity while still allowing ROS degradation. This is a general, non-specific activity tuning. This attachment can be highly stable. In preparing for the lipid coating (2), the hydrocarbon converts CeNP to a hydrophobic surface, allowing lipids to encapsulate it.

The hydrocarbon layer is a modification in preparation to encapsulate CeNPs in a lipid layer. The lipid layer increases the biocompatibility and circulation time of the particles. To perform this protocol, the hydrocarbon modified CeNPs are added to lipids in an organic solvent. The solvent is then removed and dried under vacuum to form a lipid and CeNP film. The addition of buffer promotes swelling of the lipids and they spontaneously form bilayers in response to the aqueous environment. The initial lipid-CeNP liposomes are frequently large (more than 100 nm) and multilayer instead of a single layer of lipids with a single particle core. To separate the liposomes into single CeNPs with individual lipid layers, the lipid-CeNP solution is sonicated in a water bath. This protocol has successfully produced lipid modified CeNPs using DPPC and DOPC (DPPC, 1,2-dipalmitoyl-sn-glycero-3-phosphocholine; or DOPC, 1,2-dioleoyl-sn-glycero-3-phosphocholine).

The lipid protocol is monitored using dynamic light scattering (DLS, see FIG. 14). In FIG. 14 (A), the CeNP starting material with the polyacrylate ligand only (‘bare’) has one major peak (99% of mass) centered at 0.77±1.0 nm. After lipid encapsulation and sonication, the DLS shows three major peaks (FIG. 14(B)). About one third of the population has formed the vesicles in the desired size range (4-8 nm). This population includes only those vesicles that contain a single nanoparticle core. The peaks with diameters of 350 nm (27% of mass) and 1670 nm (40% of mass) are single layer liposomes with CeNPs embedded in their lipid bilayer and multilayer liposomes. These formulations are expected to contribute to nanoparticle activity as well.

A lipid dye is also easy to incorporate into the lipid layer during the lipid modification protocol. To confirm that lipids have encapsulated the nanoparticles, fluorescence is used to show close proximity of lipids to CeNPs (see FIG. 15). In the Ce⁺³/Ce⁺⁴ ionic form, Cerium is fluorescent with an excitation peak ˜350-400 nm and an emission peak at 470 nm. When this fluorescence is coupled with a lipid dye that excites at 460-500 nm, the fluorescence should shift the lipid dye emission (520 nm). If the lipid dye and CeNPs are in close proximity, the CeNP fluorescence peak will be red shifted from 465 to 520 nm. In FIG. 15, the emission spectrum of the unmodified CeNPs is compared to the lipid encapsulated CeNPs with a lipid dye. The unmodified CeNP trace shows the intrinsic fluorescence of the CeNP using an excitation peak at 400 nm. The emission peak of ˜460 nm is visible. After encapsulating the nanoparticle with a lipid layer that includes a lipid dye of the appropriate matching excitation and emission, the nanoparticle fluorescence at ˜460 nm excites the lipid dye and the emission peak is shifted to ˜520 nm. Once again, the wetting properties of the particles change from hydrophobic (hydrocarbon modified) to hydrophilic (lipid modified).

In our second approach, we used hydrocarbon linker as a potential conduit for electron shuttling. Hydrocarbons capable of forming stable radicals that extend the electron transfer range are attached to the radius of the hydrocarbon linker. The energetics of radical formation were used as a guide to predict potential candidates. A functional group or hydrocarbon is classified as a favorable candidate if the free radical formation energy is less than +100 kJ/mol. (see FIG. 10). The free radical formation and electron shuttling of the hydrocarbon linker extends the range of the CeNP anti-oxidative activity. To promote electron shuttling, the attachment of large conjugated systems such as a series of fixed benzyl rings, like napthalene derivatives, or hydrocarbons with alternating double bonds enable the transfer of electrons to and fro the CeNP surface, promoting radical scavenging activity well beyond the nanoparticle surface. The particle characteristics are tuned by choosing a stable structure (no cleavage), which ensures high particle stability over time or cleavable functional groups, which limits anti-oxidant activity until the encapsulated CeNPs penetrate into tissues where free radicals are formed, but also allows a burst of CeNP activity in the presence of free radicals. For cleavable (unzipping) capabilities, functional groups are targeted with bond dissociation energies of less than +500 kJ/mol. Steric hindrance may or may not limit fully covering the CeNP with electron shuttling functional groups. Where full coverage is prohibited by steric hindrances, the remaining open surface sites can be filled with an appropriate amine hydrocarbon.

To increase biocompatibility and optimize circulation time, the CeNP surface can be terminated in a lipid shell. Phosphatidyl choline lipids can be used as a generic, non-reactive lipid. Phospholipids are a major class of lipids in cell membranes and the choline groups are neutral head groups that do not participate in cell signaling. In addition, phospholipids form numerous variations through choice of specific tail length, conjugation and headgroup. These include but are not limited to phospho choline (PC) lipids, phospho ethanolamine (PE), phospho thioethanol (PTE) and PEG functionalized lipids. PE and PTE are two examples of lipids useful for attachment of targeting molecules. Sphingolipids, similarly, offer a biocompatible lipid layer with various combinations of head and tail groups. Sterols, such as cholesterol, are the third major lipid family and the mixture of phospholipids, sphingolipids and sterols allows the particle lipid layer to match virtually the membrane composition of any cell type. All possibilities are viable CeNP lipid layer modifications. The lipid layer gives a biocompatible outer shell, which prevents biofouling. This lipid layer may increase circulation time of the CeNPs. In addition to hydrocarbon addition, the lipids provide a second opportunity to tune the nanoparticle activity level generically. Long chain, large lipids will increase the distance between ROS and the CeNP surface, thereby decreasing activity, while short lipids will allow for greater accessibility and activity.

The lipid encapsulation step also provides a high degree of control to tailor the surface to a specific target. Lipids enable the attachment of targeting molecules, via reactive lipid head groups, such as peptides or small molecules like L-DOPA (See FIG. 7). From this biocompatible outer layer, the invention provides the capacity to tailor the nanoparticle for a specific disease or tissue application. Specific disease or application: both dopamine, its derivatives and L-DOPA provide targeting to the brain for therapeutic efficacy to Parkinson's. Serotonin and acetylcholine are both ligands to receptors in the pancreas and can be useful for delivering CeNP for diabetes II applications.

In addition, by incorporating PEG groups in the lipid head group position, circulation time can be further increased and protein fouling can be decreased.

As explained herein, the present invention provides at least five possible points of modification within the surface chemistry strategy for CeNP tailoring, listed in order of distance from the cerium oxide particle: (1) ligand shell (the inner most linkage to the CeNP); (2) hydrocarbon additions; (3) electron shuttling (stable free radical formation) embedded in the hydrocarbon; (4) lipid shell (the outermost linkage of the hydrocarbon to the lipid shell); and/or (5) targeting molecule attachment via lipids. The CeNPs modified by these strategies result in nanoparticles tailored for tissue and organ-tailored biodistribution. These nanoparticles become increasingly reactive as the diameter decreases until ˜5 nm, where they reach their maximum ROS scavenging activity. CeNPs with ligands only, such as EDTA, citric acid or polyacrylate, are the most potent. However, without the lipid layer, they have limited circulation lifetime and poor biocompatibility (30, 31). Moreover, a benchmark test, the TPA and Fenton's reagents assays shown in FIG. 17 illustrate the activity of these particles. A unique feature of these antioxidant nanoparticles is that they can be applied multiple times: over weeks, cerium(IV)-rich particles slowly return to their starting cerium(III) content. In nearly all cases, the particles remain colloidally stable (e.g., non-aggregated) and could be applied multiple times as antioxidants. These chemical properties were also observed in cell culture, where the materials were able to reduce oxidative stress in human dermal fibroblasts exposed to H₂O₂ with efficiency comparable to their solution phase reactivity reactivity.

So to this point, the invention has detailed multiple points of modification with varying degrees of modification. As will be appreciated from the invention, all or any combination of these described modifications can be employed to achieve different characteristics depending on the needs, e.g., therapeutic, diagnostic, marking, research, etc. Without limiting possible combinations of modifications, the following is offered as examples of such modifications that are attainable for differing needs:
1. Ligand layer and hydrocarbon addition modifications;
2. Ligand layer, hydrocarbon addition, lipid shell and targeting molecule attachment via lipids;
3. Ligand layer, hydrocarbon addition, electron shuttling (stable free radical formation) embedded in the hydrocarbon, lipid shell, and targeting molecule attachment via lipids.

The invention is described in further detail through the additional embodiments provided herein.

(1) In another embodiment, formulations consist of building of the ligand shell on top of the CeO₂ surface. The application specific CeNPs are tailored via the ligand shell to specify the chemical modification. To form a ligand shell, a chelating small molecule is added during the synthesis process. It enhances stability and gives additional control over particle size (51-53). Sigma Aldrich has over 1800 carboxylic acid compounds in its catalogue. The present invention is exemplified using citric acid. However, potential surface modification strategies can be expanded using alternative chelators to create new surface chemical options.

In the case of cerium oxide, carboxylic acid chelators, such as citric acid and EDTA (Ethylene diamine tetraacetic acid), EGTA and their derivatives (115 compounds were identified) effectively bind to the surface and lead to the surface stabilization that is required for further modifications to add specific tissue targeting capability into the invention's liposomal formulations. Carboxylic acid compounds with non-reactive terminal groups (for example oleic acid) create the desired effect via non-specific modification such as lipid hybrid bilayer. See FIG. 1 and FIG. 2. Citric acid and its potential for further modification are explored in the formulations of the invention, as shown in FIG. 1, as it has three carboxylic acid (—COOH) groups, of which at least one is available to attach targeting molecules like dopamine or L-DOPA. The carboxylic acid residue of citric acid (or other organic acids used to prepare the surface of the CeNPs) can reactive with groups such as amines (—NH2), thiols or sulfhydryl (—SH), azide (—N3) or alkynes (—C═C—H) to allow the inventors further modifications under mild conditions. From these reactive handles, the CeNP surface is modified further through several reaction pathways, to tailor formulations for targeting disease-specific tissue areas. Presently, CeNPs have been successfully modified using carboxylic acid and amine coupling chemistry via EDC/NHS (see FIG. 11). Decyl amine (CH₃(CH₂)₈NH₂) reacts with the polyacrylate ligand to form an amide bond. Specifically, to produce modified CeNPs, a solution of CeNPs with a polyacrylate ligand shell is mixed with EDC and NHS (1-Ethyl-3-[3-dimethylaminopropyl]carbodiimide; N-hydroxysulfosuccinimide). Excess of the desired amine is added to the reaction mixture and allowed to react for several hours. To separate the product from the reaction mixture, an organic extraction is performed. Citrate solution is then added to remove the unreacted amine and separated in an aqueous wash.

The reaction pathways include hydrocarbon addition(s): carboxylic acid chelators, such as citric acid, effectively bind to the surface of CeNPs. Citric acid, as an example, is focused on because it has three carboxylic acid (—COOH) groups, and at least one of which is available to attach targeting molecules. The exposed carboxylic acid group enables the use of carbodiimide and succinimide chemistry (EDC/NHS) to couple the citric acid to an appropriate amine (—NH₂) terminated small molecule. This strategy mimics the peptide bond formation and is widely used to couple carboxylic acid moieties to amines (54-56). In our work, the CeNP coupling to decyl amine is tracked using infrared (IR) spectroscopy in FIG. 12 (A-C). In FIG. 12(A), the IR spectrum of the CeNP with the polyacrylate ligand shell (‘bare’ or stock CeNP) shows distinct C═O (1632 cm⁻¹) and C—O (1562 cm⁻¹) and a broad O—H stretch at 3500 cm⁻¹. The spectrum of CeNP-decyl amine (crude extract) has added C—H (2850-2950 cm⁻¹), C—O C═O and C—N bands (1300-1700 cm⁻¹). The N—H stretch (3350 cm⁻¹) indicates unreacted decyl amine, which will be subsequently removed. When the crude extract is compared to the decyl amine in FIG. 12(B), decyl amine has C—H stretched from 2850-2950 cm⁻¹, N—H stretches at 3333 and 3185 cm⁻¹ and a series of stretches and wags from 1650 to 1385 cm⁻¹, and is compared to the CeNP-decyl amide (crude extract), which shows C—H, N—H and C—N band. While the N—H stretch is still present (3500 cm⁻¹) in the crude extract, it is comparatively smaller than the decyl amine N—H stretch. As well, the C—O, C═O and C—N have all shifted in the 1300-1700 cm⁻¹ region. After adding citrate to the crude extract shown in FIG. 12(C), the unreacted amines then form amide bond with the citrates and partition into the aqueous phase, leaving behind separated product. The amine N—H stretches at 3333 cm⁻¹ disappears compared to the crude extract. As well, the amide carbonyl (1700 cm⁻¹) and C—N (1650 cm⁻¹) stretches are more prominent.

Using nuclear magnetic resonance (NMR) spectroscopy, the unreacted decyl amine is compared to the CeNP product. The decyl amine terminal methyl and interior methylene peaks are observed at 0.88 and 1.27 ppm δ ¹H (chloroform, CDCl₃). The β-methylene protons, two carbons away from the amide bond, remain unshifted at 1.45 ppm. After the reaction, the α-methylene protons (those adjacent to the amide bond) shift from 2.68 to 2.21 ppm. A weak peak at 7.85 ppm appears, which is attributed to the amide proton peak. The single amide hydrogen has exchanged with deuterated solvent (CDCl₃) to give a merely a small blip. In this example, there is some unreacted amine (overlapping peak at 2.68 ppm).

In addition to confirming the amide coupling using IR and NMR spectroscopies (see FIGS. 12 & 13), the nanoparticles partition into the organic (chloroform, di-ethyl ether, hexanes, or other organic solvent) phase during the separation and purification steps. The hydrocarbon exterior has transformed CeNPs from hydrophilic to hydrophobic particles.

(2) Sigma Aldrich offers ˜5000 amines in its catalogue to attach to the exposed citric acid ligand. Outlined herein are a few examples. Using well-characterized reactive groups, there is a wealth of surface modifications.

Unzipping (labile) bond(s) embedded within the encapsulated CeNP formulation: To ensure efficacy, the surface of the CeNP is modified to incorporate labile bond(s) that is/are susceptible to free radical attack and cleavage from to the lipid shell. The oxidatively damaging environment (high concentrations of free radicals) cleaves the labile bond, unzipping the lipid/PEG surface to expose the CeNP and maximize its anti-oxidant activity (Trojan strategy). Such a strategy is not known to be used with diagnostic and therapeutic applications of anti-oxidants. This gate-keeping approach is embedded within the invention's design in such a way as to allow unzipping of the CeNP from the lipid hybrid layer to release the active agent at its target and allow the CeNP maximum effective activity in a cellular location that optimizes its therapeutic or diagnostic action. This is achieved, in part, by choosing hydrocarbons with functional groups that form stable radicals (such as acetyls, or ethers (such as acetals, epoxides, amides, peroxides or ethers (see FIG. 10 and (47)).

(3) FIG. 5 demonstrates the method of preparation of such a particle with Trojan strategy delivery and FIG. 6 shows the mechanism of unzipping and action intracellularly. The previously unexplored modification of the particle surface chemistry allows embedding a cleavable bond that is cleaved inside the target cell, to allow the formulation to shed the layers that do not contribute to the therapeutic action of CeNPs and expose the therapeutic/diagnostic CeO₂ particle. The methods proposed for engineering these formulations allows multiple permutations with a range of surface coverage (from less than 1/10 coverage to complete coverage) to vary accessibility to the free radical cleavable bond. All these permutations result in disease and tissue specific formulations that are tailored to be effective in various chronic disease situations.

In another aspect, electron shuttling capabilities are provided. To ensure efficacy, the surface is modified to incorporate functional groups that form stable free radicals to create a larger radius of CeNP antioxidant activity while creating a surface compatible to the Lipid Shell. Controlling the range of radical interactions with CeNP (from short to long), a variety of formulations can be created that encompass all applications for long term dosage in a variety of chronic inflammation disease, with low toxicity profile and for maximized therapeutic or diagnostic potency. These formulations bring CeNP and radicals together for action both through near contact and extended contact range.

To verify the radical scavenging activity, the nanoparticles are tested using Fenton's reagent. Specifically, CeNPs are mixed into a solution of Iron (II) (Fe⁺²) in ammonium chloride solution, and subsequently, a small amount of hydrogen peroxide (H₂O₂) is added. The hydrogen peroxide will slowly convert Fe⁺² to Fe⁺³ (Fe²⁺+H₂O₂+H⁺→Fe³⁺+HO.+H₂O) as hydrogen peroxide degrades. When CeNPs are added, the conversion of Fe⁺² to Fe⁺³ increases since cerium oxide will act as an oxidation/reduction partner, cycling from Ce⁺⁴ to Ce⁺³ (see scheme 1: 2Ce⁺³+3O⁻²+2HO.→2Ce⁺⁴+4O⁻²+H₂O). After a designated amount of time, 1,10-phenantholine (PA) is added to the reaction solution. It forms a complex with Fe⁺² and has a strong color change (colorless to bright red, absorbance at 520 nm, see FIG. 16(A)-(B)). The concentration of Fe⁺² is measured using visible spectroscopy and compared to ascertain the activity of the CeNPs due to their modifications.

As shown in FIG. 17 (A-C), the data indicate that the sample with the most Fe⁺² is the control assay without any H₂O₂ and the entire amount of free Fe⁺² in solution forms the bright complex. The Fe⁺²/PA assay establishes the maximum absorbance possible under these conditions, shown in FIG. 17(B). When H₂O₂ is added to the Fe⁺² solution, in the reaction time, a portion of the initial Fe⁺² present converts to Fe⁺³ as shown in the Fe⁺²/H₂O₂/PA trace (FIG. 17 (A)). When CeNPs are added, there is a clear increase in conversion rate from Fe⁺² to Fe⁺³ (FIGS. 17 (A) & (C)). The sample with the least amount of Fe⁺² is the unmodified CeNP (Fe⁺²/H₂O₂/Unmodified CeNP/PA). It has the least 1,10-phenantholine +Fe⁺² complexes and the lowest absorbance at 520 nm, since it has converted the most Fe⁺² to Fe⁺³. While it has the highest activity, it is also the least biocompatible. Nanoparticles with a mixed hydrocarbon layer of low coverage of acetal amides (from 2-(1,3-Dioxolan-2-yl)ethanamine) and high coverage of decyl amide exhibit definite anti-oxidant activity. CeNPs with full decyl amide have a lower activity level. The difference between the acetal/decyl and full decyl amide modified CeNPs alone (FIG. 17 (D)) is expected. The acetal amide will cleave in the presence of radicals (see FIG. 10), like those generated by hydrogen peroxide. The cleaved acetal group acts as an unzipping agent, and the acetal modified CeNPs shed their lipid layer. Due to the cleavage, hydroxyls form proximate to the cerium oxide, and the new functional groups change the nanoparticle wetting behavior from hydrophobic to hydrophilic. Depending on the ratio between acetal to decyl amide, the activity level can be tuned.

Using the calibration curve of Fe⁺² concentration (FIG. 18), the activity data is converted into Fe⁺² concentrations and those numbers give a quantitative comparison of CeNP activity. Beer's Law states that there is a direct relationship between absorbance and the absorbing species: A₅₂₀=bcε₅₂₀, where A is absorbance at 520 nm; b is path length of the cuvette; c is concentration of the absorbing chemical species; ε₅₂₀ is the molar absorptivity of the absorbing species). Since both path length (b) and molar absorptivity are held constant, the linear relationship between absorbance and concentration of Fe⁺² is easily established.

From the Fe⁺² calibration curve, the activity assay is quantified (see FIG. 19). From an initial Fe⁺² value of 299 μM, the maximum absorbance value from the Fe⁺²/PA control run gives an upper limit of ˜220 μM. The Fe⁺²/Fe⁺³ reach equilibrium at pH 5 corresponding to ˜220 μM/˜80 μM. For the activity assays with H₂O₂ present, the inflection point represents the freely available Fe⁺² that forms a complex with PA readily. All inflections points are estimated to occur at 15 s after PA is added. When H₂O₂ is added to the Fe⁺² solution, approximately 88 μM of Fe⁺³ is converted. In contrast, the decyl CeNP increases the amount of Fe⁺³ to 126 μM, corresponding to an increase of 38 μM. Acetal CeNP converts 150 μM, or 62 μM higher. The unmodified CeNP produces the highest amount of Fe⁺³ in this assay of ˜200 μM.

The data (see FIGS. 17-19) indicate that the most reactive nanoparticle, which has converted the most Fe⁺² to Fe⁺³, is cerium oxide with only the polyacrylate ligand—the unmodified or ‘bare’ CeNP. However, it is also the least biocompatible. Nanoparticles with a mixed hydrocarbon layer of low coverage of acetal amides (from 2-(1,3-Dioxolan-2-yl)ethanamine) and high coverage of decyl amide exhibit definitive activity, increasing Fe⁺³ production by 62 μM more than was produced when CeNP was not present. CeNPs with full decyl amide have a lower activity level. The difference between the acetal/decyl and full decyl amide modified CeNPs alone is expected. The acetal amide will cleave in the presence of radicals (see FIG. 10), like those generated by hydrogen peroxide. The cleaved acetal group acts as an unzipping agent, and the acetal modified CeNPs shed their lipid layer. Due to the cleavage, hydroxyls form proximate to the cerium oxide and the new functional groups change the nanoparticle wetting behavior from hydrophobic to hydrophilic. Depending on the ratio between acetal to decyl amide, the activity level of CeNP can be tuned.

Once unzipped, the cerium oxide surface becomes accessible to the oxidatively damaged tissue of various origins or the targeted tissues, depending on the disease being diagnosed and/or treated.

In another embodiment, the CeNP surface is encapsulated in a lipid or polyethylene glycol shell resulting in lipid/PEG hybrid bilayer, illustrated by FIG. 3. While the ligand shell also affords the opportunity to attach a long hydrocarbon chain, producing a hydrophobic nanoparticle, the use of five modification points maximizes the tailoring opportunities. The lipid shell (polysorbate (Tween) surfactants, Lactate, Apolipoprotein—E, amidation) offers an opportunity to attach peptides or small molecules, such as L-DOPA, dopamine, serotonin, acetylcholine and their derivatives and targeting proteins, e.g. transferrin.

(4) To further tailor particles of the invention. When combined with lipids and PEG modified lipids, the result produces a hybrid bilayer in an aqueous environment. The functionalization of the invention's formulations is demonstrated in FIG. 4 of the attachment.

The application of polyethylene glycol modified lipids increases the circulation lifetime of liposomes (57). Lipid and PEG modified devices, drug filled liposomes increase biocompatibility and decrease protein fouling (58). The invention takes advantage of both of these aspects to maximize the CeNP efficacy. From the lipids and PEG lipid options (http://avantilipids.com), the invention tailors the encapsulated CeNP for specific tissue and disease applications.

In another embodiment, the formulations include targeting molecules to tissue-specific delivery, as described herein. In another embodiment, methods of attaching a variety of application-specific peptides are used. It is recognized that peptides offer a rich pool of future targeting molecules. As such, while the C-terminus and N-terminus provide the necessary reactive groups to attach a peptide, the free thiol of cysteine provides a direct, tailored linkage to the invention's nanoparticle. This reaction pathway is exploited using small molecules that couple carboxylic acids and thiols such as amine maleimides that are available through Sigma Aldrich. The amine portion will react with carboxylic acid surface while the maleimides react with thiol. Alternatively, small molecules with amine and thiol groups result in a di-sulfide linkage. While the amine group couples with the (e.g., citric acid) ligand, the exposed thiol groups readily react with free thiols in solution under mild conditions. The reaction conditions are adjusted using excess peptide or by reducing the number of thiols on the surface to maximize peptide attachment and to minimize particle dimerization. This di-sulfide bond is labile and readily cleaves under reducing conditions. However, the targeting peptide is used to correctly position the nanoparticle in close proximity to the oxidative damage. Once at the location, it is the cerium oxide that provides treatment to the damaged cells and tissue. The peptide is not the therapeutic agent.

For tissue/cell targeting, the attachment of peptides is not limited to traditional biomolecular labeling. Embedding azides and alkynes though specialty amino acids opens the door to click chemistry attachment.

In another embodiment, the formulations are engineered to maximize the biocompatibility while on route to the targeted tissue and then unleash the active action intracellularly, once delivered to and unzipped in the disease tissue. This hybrid bilayer takes advantage of the lipid and PEG properties to maximize biocompatibility and circulation time in vivo. However, such a layer has also been used to passivate reactive inorganic surfaces (59-63). To ensure efficacy, the surface is modified to incorporate a labile bond prior to attachment of the hybrid bilayer. The oxidatively damaged environment will cleave the bond, unzipping the lipid/PEG surface to expose the cerium oxide particle and to maximize its activity (Trojan strategy). Such strategy is not known to be used with diagnostic and therapeutic applications of anti-oxidants. This gate-keeping approach is inherent in the invention's design of the particle surface so as to allow the removal of the drug or the lipid hybrid layer and release the active agent at its target to allow the CeO₂ maximum opportunity to effect its therapeutic or diagnostic action. This is achieved by choosing hydrocarbons with functional groups that form stable radicals (such as acetals, epoxides, amides, peroxides or ethers (see FIG. 10 and (47)). Once unzipped, the cerium oxide surface becomes accessible to the oxidatively damaged tissue of various origins, depending on the disease being diagnosed and/or treated.

In another embodiment, the formulations carry the additional layer modification by coupling ligands and homing devices for tissue penetration and specific organ uptake via receptor recognition process via receptors selectively or semi-selectively expressed by the tissues involved in the pathogenesis of CNS, pulmonary, autoimmune and amyloid disorders, variety of cancers. Specifically, for COPD applications, there will be attached, for example, long-acting anticholinergics (such as tiotropium bromide), acetylcholine, long-acting muscarinic antagonists, with functional and kinetic selectivity for muscarinic receptors M1, M3 and M4. An example of such a ligand is scopolamine. Another class of receptors for targeting with present formulations is beta-adrenoceptors in human airways. Specifically, for CNS disorders coupling ligands targeting serotonergic systems are attached (for example, 5-HT1A receptor ligands: serotonergic type 1A (5-HT 1a) receptor agonist, serotonergic type 2A (5-HT 2a) receptor agonist). Other ligands utilized are buspirone, sarizotan, tandospirone. Specifically, for Parkinson's disease applications ligands to metabotropic glutamate receptors (mGluRs) are attached to the formulations (example include both positive allosteric modulators of mGluR2 and mGluR4; and negative allosteric modulators of mGluR5). NDMA receptor antagonist to selectively target NR2B subunit and antagonist of the metabotropic glutamate receptor mGluR5.

Another embodiment is the attachment of neurotransmitters (such as L-DOPA and 6OHDA) that are taken up into cells by dopamine and norepinephrine reuptake transporters. Incorporated are analogues/congeners of neurotransmitters and proteins or parts of proteins into the liposomal coating that are transported across the cell membranes by specific transporters or transported with special affinity by virtue of the lipid solubility of the liposomal coat or transported by virtue of the long circulation time associated with PEGylation or other molecular modifications of the liposomal coat that prevent or limit uptake by the reticulo-endothelial system.

Ligands used in the present invention's formulations include but are not limited to: oleic acid, Insulin, IGF-1 IGF-2, leptin, transferrin, L-DOPA and dopamine FIG. 7 describes the series of strategies that can be employed to add such various attachments to the invention's formulations. Specific example of how the above-described modifications are applied to tailor formulations for action in specific disease is demonstrated in FIG. 8, which illustrates the methodology to use to attach L-DOPA to the lipids outside of the shell for expected activity in CNS diseases. Strategies for specific targeting of L-DOPA CeO₂ particles to midbrain dopamine neurons is based on selective expression of dopamine-transporter (DAT) by these brain cells (64). These DAT-expressing midbrain neurons are highly susceptible to ROS and oxidative stress, loss of which leads to Parkinson's disease (65, 66). Following the selective uptake, in the reducing environment of the cytosol, the particle will shed its lipid coat allowing access of CeO₂ to ROS and their quenching. Based on the particle anti-aggregation properties, also it is also expected that local accumulation of the invention's CeO₂ nanoparticles in tissues expressing amyloid peptides such as the brain and pancreas will reduce the extent and/or rate of amyloid-induced 1-oxidative stress in these two amyloid-sensitive tissues. Thus, direct inhibitory effects of CeO₂ nanoparticles on ROS production and accumulation in amyloid-burdened tissues can be achieved.

In another embodiment, these CeNP compositions can be in intranasal, oral, inhaled, eye drops and parenteral formulations. The formulations can be determined based on the use and requirements of the disease or condition be treated or the diagnostic test being utilized.

(1) Dried: After modifying CeNPs on the ligand level, they may be dried down and re-hydrated. This method must be tested to ensure proper dispersion.
(2) Liquid: After adding a lipid layer, CeNPs must be kept in solution. Lipids are readily oxidized after drying. Repeated cycles of hydration and drying leads to lipid degradation. Liquid forms of delivery include IV application or possibly injections.
(3) Aerosol: Naked, ligand and surface modified CeNPs and liposomal variations of CeNPs are all amenable to aerosol applications. The liquid droplets are sufficient to keep the lipids hydrated. This formulation allows for nasal spray and lung inhalation applications.
(4) Gel capsule/Oral delivery: For oral applications, the modified CeNPs must pass though the digestive system. To protect the surface chemistry from the harsh environment, possibly a protective gel capsule would deliver the CeNPs into the blood stream after passing though the gut.

In another embodiment, the present invention uses homo- and hetero-bifunctional linkers. Chemical modifications described herein are based on the citric acid ligand, but introducing other chelating molecules with protected amines or other functional groups can provide new chemical avenues to explore. Formulations can also be developed when light cleaving functional groups are added to effectively shed the outer lipid layers. See (58).

In another embodiment, the diagnostic applications of CeNPs are exploited with the intrinsic fluorescence of the particles and chemical attachment of fluorescent dyes to the CeNP surface, as described in FIG. 9. In the +3/+4 state, cerium has fluorescent properties (350 nm excitation/460 nm emission, see FIG. 15) (67).

(1) In the naked or encapsulated with a ligand shell, its emission may be coupled with an appropriate short wavelength dye (400-550 nm, www.probes.com). When the CeNP and the dye are in close proximity (>10 nm), detection of an energy transfer event (FRET) is expected, which will confirm colocalization. Such a tool is useful in cell studies, tagging the plasma membrane or specific parts of the cell with the dye to track CeNP interactions. In determining lipid proximity, it has proven useful to show the lipids and CeNP are within 10 nm.
(2) As well, dyes can be attached to the carboxylic acid surface of CeNPs using an amine terminated dye derivative. Alternatively, other terminal reactive groups of the dyes may be used with appropriate CeNP surface chemistries. From the FRET events, diagnosis of the loading of the dye to the surface and use of such chemistry as a characterization tool is expected.
(3) While short wavelength dyes offer FRET capabilities, pairing the CeNP surface with longer wavelength dyes will allow characterization of the ratio of CeNP to dye molecules, providing loading information as well. As mentioned previously, dye derivatives with amine or other reactive terminal groups provide a chemical means to attach them to the CeNP surface. Additionally, longer wavelength dyes are more compatible with cells, since excitations in the 250 nm range carry the potential to cause photo-damage. Again, tagging different parts of the cells with an appropriate FRET dye counterpart will detect interactions within ˜10 nm of CeNPs, proving colocalization.
(4) Through the use of fluorescent dyes on CeNPs, the capabilities exist to track in vivo circulation and quantify their uptake into the targeted tissue or to off-target sites as well as CeNP concentration through fluorescence microscopy and fluorometry.
(5) Finally, to test the unzipping mechanism in vitro, a lipid dye can be incorporated into the bilayer. When CeNPs are exposed to a reducing environment, the fluorescent signal of the particles and the lipids can be monitored. Detection of only CeNP signal after washing the particles in buffer is expected.

The present invention overcomes the short comings of previously used antioxidants. Firstly, the invention's formulated CeNP particles localize to affected tissues (for example, cross the blood brain barrier), becoming effective therapy for the diseases in which excess ROS play an important role. Using the available carboxylic group from citric acid, hydrocarbon linkers, lipids and targeting molecules can be attached. In this manner, maximization of uptake in targeted tissues can be achieved, and anti-oxidant activity can be controlled (passivated during drug delivery) and unmasked at tissues sites with high concentrations of ROS and/or RNS. The effect of small molecule accessibility is created by varying the distance of targeting molecule from the cerium oxide or lipid surface. The lipid hybrid layer provides a proven strategy to decrease protein fouling and increase circulation via PEG lipids.

Secondly, the compound must accumulate in the affected tissues at a high enough concentration to be clinically effective in the treatment of the disease. In case of the CNS diseases, fewer than 2% of ‘small molecule’ drugs are capable of penetrating the blood brain barrier, and only a fraction of these have appreciable deposition in the brain (23, 24, 68). Thus, CeNPs can be dosed infrequently and still achieve appreciable tissue levels compared to other antioxidants (69-73). By introducing novel nanoparticle surface modifications, such as the unzipping layer or electron shuttling capabilities and embedding a weak linkage or a functionality within the liposome, the particle properties can be tuned for maximum biocompatibility and targeting. The problem of lipid layer passivation can be circumvented by incorporating a labile bond that will cleave in the presence of ROS or extending the redox activity of CeNP and/or introducing cleavable or shedding layers to the CeNP.

In one embodiment, once doubly unzipped, the CeO₂ surface becomes accessible to the oxidatively damaged tissue. Improvements in cell penetration, deposition and intracellular reactivity will significantly improve the pharmacokinetic properties of CeNPs when used in vivo.

In another embodiment, the CeNPs passivated by our methods become tailored for tissue and organ-specific biodistribution with pharmacokinetic profiles that minimize systemic toxicity by avoiding uptake by the liver and spleen, minimize redox reactivity in tissues not involved in the disease pathology and maximize anti-oxidative effect at the site(s) of greatest oxidative stress.

As will be appreciated, the present invention provides multiple points of modification of the CeNPs. In one embodiment, the modifications serve to passivate the CeNP redox activity, partially to completely. For example, in addition to long hydrocarbons (2-40, 4-20, 6-12, 8-10 carbons and/or longer), bulkier side chains (e.g., a tert-butyl group, cycloalkanes, dendritic structures, polypropylene functionalities) in the middle of the hydrocarbon chain, and other functional groups, such as fluorinated derivatives and polymer structures, are all candidates to block or interfere with the activity of the CeNP. In one embodiment, a multi-layered and passivated or “off” formulation of CeNPs can be created. Using the surface modification strategies described herein for CeNP tailoring, the CeNP redox activity can be partially or completely passivated. The passivated CeNP are tailored to improve tissue and organ-specific uptake.

In another embodiment, these engineered nanoparticles shall be used as diagnostic agents for diagnosis and prevention of chronic diseases such as: systemic illnesses such as COPD-emphysema, asthma, Idiopathic fibrosing pancreatitis (IFP); systemic autoimmune disease such as type-1 diabetes, arthritis and degenerative amyloid-induced brain and pancreatic diseases such as Alzheimer's, Parkinson's, Glaucoma, Macular Degeneration, Traumatic Brain Injury, Cardiovascular diseases and type-2 diabetes mellitus, in which oxidative stress and/or amyloid formation play a pathological role (38, 48-50).

Lastly, the therapeutic agent must have a long half-life sufficient to neutralize excessive amounts of ROS produced as part of chronic disease process. CeNPs have a long half-life in tissues, which allows novel dosing schedules. One may use CeNPs to ‘vaccinate’ individuals who may be at high risk of oxidative stress in the future (e.g., individuals susceptible to head trauma and traumatic brain injury, such as soldiers or athletes in contact sports). The application of polyethylene glycol modified lipids increases the circulation lifetime of liposomes (57). Lipid and PEG modified devices, drug filled liposomes increase biocompatibility and decrease protein fouling (58). Both of these aspects can be taken advantage of to maximize the CeNP efficacy. From the lipids and PEG lipid options (http://avantilipids.com), the encapsulated CeNP can be tailored for specific tissue and disease applications. Lipid encapsulation and PEGylation increase the circulation time and increase targeted tissue uptake—thereby prolonging the tissue half-life of the CeNPs.

The following is an exemplary list of chronic illnesses with strong established roles of RS in pathogenesis that are subject to the instant invention: Parkinsons Disease; Alzheimers Disease; Amyloidosis; Demential with Lewy Bodies; Neurodegeneration with Brain Iron Accumulation Type 1; Other adult-onset basal ganglia diseases; Non-Alzheimer's Tauopathies including: a) Pick's disease (frontotemporal dementia), b) Progressive supranuclear palsy although with straight filament rather than PHF tau, c) Dementia pugilistica (chronic traumatic encephalopathy), d) Frontotemporal dementia and parkinsonism linked to chromosome 17 however without detectable β-amyloid plaques, e) Lytico-Bodig disease (Parkinson-dementia complex of Guam), f) Tangle-predominant dementia, with NFTs similar to AD, but without plaques (tends to appear in the very old), g) ganglioglioma and gangliocytoma, h) Meningioangiomatosis, i) Subacute sclerosing panencephalitis, j) As well as lead encephalopathy, tuberous sclerosis, Hallervorden-Spatz disease, and lipofuscinosis, k) Frontotemporal dementia, and l) Frontotemporal lobar degeneration; Amyotropic Lateral Sclerosis; Traumatic Brain Injury and Chronic Traumatic Encephalopathy; Spinal muscular atrophy; Spinocerebellar atrophy; Multiple Sclerosis and Idiopathic Inflammatory Demyelinating Diseases; Chronic Inflammatory Demyelinating Polyneuropathy and other autoimmune demeylinating diseases; Periventricular leukomalacia and cerebral palsy; Creutzfeldt-Jakob (prion) disease; Friedreich's Ataxia; Hallervorden-Spatz disease; Muscular Dystrophy; Huntington's; Vascular dementia; Cerebral ischemia; Cardiovascular disease and plaque formation, myocardial infarction and re-perfusion injury; Myocarditis; Cardiomyopathy; Stroke: reperfusion injury following hypoxia; Diabetes; Glaucoma; Age-related macular degeneration; Cataracts; Hearing loss; Chronic renal failure; Glomerulonephritis; Liver cirrhosis, alcohol liver disease, hepatic fibrosis; liver ischemia and reperfusion injury; AIDS-related dementia and HIV encephalitis; Septic shock and organ failure; Sickle cell disease; Inflammatory rheumatoid and osteo-arthritis; Rheumatoid arthritis; Aging; Bacterial meningitis; Necrotizing entrecolitis; Celiac disease; Inflammatory bowel diseases—Crohn's, ulcerative colitis; Systemic lupus erythematosus; Atopic dermatitis—eczema; Chronic obstructive pulmonary disease (COPD) asthma, emphysema (numerous); obliterative bronchiolitis; Idiopathic pulmonary fibrosis, idiopathic interstitial pneumonia (IIP), which is in turn a type of interstitial lung disease; Acute lung injury; Septic and distressed lung (respiratory distress syndrome); Inclusion body myositis; Carcinogenesis; Acne vulgaris; Epilepsy; Depression; Anxiety; Bi-polar disorder; Schizophrenia; Male infertility; Fibromyalgia; and Chronic fatigue syndrome. The diseases and disorders, as well as those discussed throughout the specification, are subject to the instant invention.

### Therapeutic Options

The efficacy of antioxidants has been widely studied in most of the major chronic diseases, including carotinoids, flavinoids, vitamins (including ascorbate and tocopherol), minerals (zinc, selenium), fruit and vegetables and extracts, ubiquinone (Coenzyme Q-10), glutathione (glutathione esters, glutiathone peroxidase mimetics, inducers of glutathione biosynthesis), lipoic acid, melatonin, thiol compounds (N-acystelyn, N-isobutyrylcysteine, synthetic novel thiols, and N-acetyl-L-cysteine), nitrone spin traps, superoxide dismutase (SOD) and catalase, SOD mimetics, and redox sensor inhibitors. Some studies have shown efficacy, but struggle to adequately control for extraneous factors, and they often fail to replicate similar studies. SOD and catalase are common and biologically effective endogenous antioxidant enzymes, but their exogenous introduction is problematic because these enzymes cannot be readily taken up by cells. Bioavailability is a significant complicating factor in exogenous antioxidant strategies generally; even if adequately located to sites of inflammation, most antioxidants are consumed in a single interaction with a free radical, limiting their scavenging effectiveness without a means of providing continuous dosing.

To be clinically effective anti-inflammatory therapies need to locate preferentially to the target organs (e.g., the CNS or lung parenchyma) and target specific cells and pathways that are quantitatively important in disease pathogenesis, preferably in a catalytic manner. The levels of antioxidants need to be sustained for long periods of time. Chronic diseases demand chronic therapies with sustained levels of effective drugs in tissues and organs.

Cerium is a transition metal, lanthanide element. Its oxide, CeO₂ (“ceria”), has a fluorite crystalline structure containing oxygen vacancies which exhibit a large diffusion coefficient. These factors combine to facilitate a reversible conversion in which Ce can exist in two oxidation states, Ce3+ (fully reduced) or Ce4+ (fully oxidized), allowing it to show both superoxide dismutase and regenerative catalase activity:

[chemical structure]

In a redox environment 5-10 nm ceria particles with high surface area-to-volume ratios have potent reactivity, are readily interchangeable between these states, and show particular reactivity/affinity for oxygen containing free radicals, making them highly effective, regenerative (catalytic) free radical scavengers of superoxide and peroxynitrite. Additionally, at nano-scale, surfaces of ceria nanoparticles have a high hydrogen and oxygen-absorbing capacity, providing for ease of reaction with H₂0₂, or H₂0 and their associated radical species. Ceria nanoparticles have demonstrated access to intracellular and intercellular spaces, penetrating to ‘protected’ environments important in inflammatory diseases. Ceria have been tested in culture and animal models with demonstrated efficacy in neutralizing RS activity and injury:
Ceria nanoparticles preserve striatal dopamine and protect dopaminergic neurons in the substantia nigra in the MPTP-mouse mode of Parkinson's disease, with dose response being bell-shaped.
Ceria nanoparticles localize, in part, to mitochondria and decrease cellular death and dysfunction associated with rotenoneinduced inhibition of complex I activity in mitochondria.
Pretreated ceria nanoparticles enter intact into endosomal compartments in human bronchial epithelial and mouse macrophage cell lines without inflammation or cytotoxicity; they suppress ROS production and induce cellular resistance to oxidative stress.
Cerium oxide nanoparticles protect a variety of cell culture systems against oxidative damage (UV light, peroxide, irradiation and glutamate induced excitotoxicity).
Treatment of murine macrophage cells with cerium oxide nanoparticles suppresses inducible nitric oxide synthase and mRNA levels in a concentration-dependent manner and quenches reactive oxygen species with no toxic effects to cells at any concentration tested.
In a murine model of ischemic cardiomyopathy cerium oxide nanoparticles markedly inhibited infiltration of monocytes and macrophages, accumulation of 3-nitrotyrosine (a marker of peroxynitrite nitration of tyrosine), apoptotic cell death, and expression of pro-inflammatory cytokines, tumor necrosis factor TNF-α, IL-1β, and IL-6.
Ceria nanoparticles protect cell viability and cell morphology of human neuroblastoma cells against amyloid-B injury in an Alzheimer's model and demonstrate neurotrophic effects.
Cerium oxide nanoparticles protect brain slices against injury in a model of ischemia and reperfusion (simulating stroke).
Cerium oxide nanoparticles protect against inflammatory cell damage induced by traumatic brain injury in an in vitro model using rat cortical microglia.
Cerium oxide nanoparticles attached to carbonic anhydrase (an enzyme) reduce oxidative retinal damage in rats.
Cerium oxide nanoparticles given in tail vein injections reduce the severity of Experimental Autoimmune Encephalitis (a model of relapsing Multiple Sclerosis).
Preliminary toxicology studies in rats found that intravenously delivered ceria nanoparticles accumulated predominantly in the most oxidative organs (brain, heart, and lung) and remained at least 6 months post-injection with no overt toxicological effects.

Ceria nanoparticles appear remarkably non-toxic in short and long duration experiments (days to weeks to months). High doses between 50-750 mg/Kg have been given with little evidence of acute systemic toxicity (33). Safety studies of CeO₂ in the U.S. and Europe have found no serious toxicity or mutagenicity. The therapeutic range will be between 0.1-500 mgs/kg depending on the route of administration. Higher doses may be given orally, middle range doses given intravenously or inhaled or subcutaneously and very low doses given intraocularly. The current intravenous and subcutaneous doses in animals range between 5-60 mgs/kg given as frequently as daily and as infrequently as weekly. Human doses will encompass the range of 0.1-100 mgs/kg given on variable schedules (daily to weekly to monthly for systemic administration and as infrequently as 3-6 months for intraocular administration) depending on clearance rats of the drug. Importantly, the protective actions of ceria particles are regenerative because their activity is catalytic and not consumed in their antioxidant reactions with peroxynitrite and superoxide radicals. The particles have a half-life measured in weeks in animals; sustained and relatively even levels of effective antioxidant activity can be achieved with regularly spaced administration intervals or a regimen that allows front loading of ceria particles into the diseased organ or tissue and then providing maintenance follow up treatments (bolus followed by boosters with prolonged withdrawals from ceria particles administration in between). Front loading can be used as a strategy to optimize the pharmacodynamic profile and regenerative nature of ceria particles through the administration of high doses early in therapy for a short duration. Front-loaded regimens may be administered over 3-90 days.

Methodology

1. Create a Potent, Bioavailable Cerium-Oxide Nanoparticle Platform with Superior Therapeutic Properties.

Ceria nanoparticles (5-10 nm diameter) possess biological activity and decreased levels of peroxynitrite and superoxide, reducing the damaging cellular effects of inflammation. Modified cerium oxide nanoparticles can be synthesized with novel coatings and carriers or multifunctional hydrocarbons, and internal modifications can be developed to enhance redox reactivity, target the particles to particular tissues and enhance penetration of the particles to sequestered environments (absorption across epithelial layers of the lung, enhance penetration of the blood brain barrier). Engineering of composition and surface characteristics, packaging and delivery vehicles that can be administered by inhalation to the lung or intravenously for selective uptake will minimize accumulation, increase bioavailability, and optimize efficacy specifically at sires of free radical generation.

2. Demonstrate the Effectiveness of Inhaled Nanoparticles in an Animal Model of Emphysema, Mitigating and Ameliorating Lung Damage Following Cigarette Smoke Exposure.

Studies can be conducted of the effects of inhaled, chemically unmodified, cerium oxide nanoparticles in two strains of rats exposed to cigarette smoke for 20 weeks at a dose equivalent to smoking 2 packs/day, testing whether administration of inhaled nanoparticles will reduce the concentration of peroxynitrite (as reflected by nitrosylation of proteins) and reduce measured severity of lung damage following cigarette smoke exposure. Rat strains vary in their susceptibility to cigarette smoke-induced lung disease. This exposure duration and intensity is sufficient to generate emphysema in approximately 75% of the animals. Two rat strains can be studied, both of which are already accepted in this field of research. The severity of lung disease functionally can be defined by measuring lung volume changes and diffusion of carbon monoxide in cigarette-exposed and control animals and in drug-exposed and control animals (two-by-two experimental design). Safety, side effects and toxicology can be assessed. Emphysema pathologically can be defined by measuring the size of alveoli in the different animal groups using stereological methods. Finally, the mechanism of action and the tissue specific loading with ceria particles can be confirmed by using a combination of lung lavage fluid analyses for cytokines and tyrosinated proteins, and lung tissue analyses to measure intracellular ceria levels in lung tissue. This work can be concurrent with #1.

3. Demonstrate the Effectiveness of Infused Nanoparticles in an Animal Model of Parkinson's Disease.

A randomized, double blinded study can be conducted in mouse models of Parkinson's disease to establish bioavailability of ceria particles across the blood-brain barrier in large numbers and efficacy in slowing or arresting progression of the disease. 6-hydroxydopamine is a recognized animal model of Parkinson's disease. 6-hydroxydopamine is a toxin that kills dopaminergic neurons by a free radical-dependent mechanism. Studies can be carried out parallel to the COPD studies outlined above in a model of Parkinson's Disease created by injection of 6-hydroxydopamine in a 2×2 design to test whether naked 5-10 nm ceria particles will reduce the severity of Parkinsonian symptoms in the treated rats, and this amelioration of symptoms/reduction of 6-hydroxydopamine toxicity would be even greater in animals treated with ceria oxide nanoparticles delivered in carrier vehicles due to increased bioavailability across the blood-brain barrier. Safety, side effects and toxicology can be assessed. This work can be concurrent with #1 and #2.

4. Demonstrate the Effectiveness of Ceria Particle-Carrier Product In Vivo in Animal Models.

Following completion of first generation carrier technology (#1 above) and indications of naked nanoparticle administration (#2 and #3 above), test safety and effectiveness of ceria-bearing carriers in the COPD and Parkinson's models, same design. Conclusion of this work constitutes proof-of-principle to justify completion of IND work and pursuit of human trials, first in COPD and following in Parkinson's. This work can be after the conclusion of #1, 2, 3 above.

The invention has been described with references to a preferred embodiment. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

### REFERENCES

The following list provides those references cited herein. To the extent these references are relied on, each is hereby incorporated by reference.
1. Beckman J S, Crow J P. Pathological complications of nitric oxide, superoxide and peryoxinitrite formation. Biochemical Soc Trans 1993; 21:330-334.
2. Hall E D, Wang J A, Miller D M. Relationship of nitric oxide synthase induction to peroxynitrite-mediated oxidative damage during the first week after experimental traumatic brain injury. Exp Neurol 2012; 238:176-182.
3. MacNee W. Oxidative stress and lung inflammation in airways disease. Europ J Pharm 2001; 429:195-207.
4. Mustafa A G, Singh I N, Wang J, Carrico K M, Hall E D. Mitochondrial protection after traumatic brain injury by scavenging lipid peroxyl radicals. J Neurochem 2010; 114:271-280.
5. Smith K J, Kapoor R, Felts P A. Demyelination: The role of reactive oxygen and nitrogen species. Brain Pathol 1999; 9:69-92.
6. van Horssen J, Witte M E, Schreibelt G, de Vries H E. Radical changes in multiple sclerosis pathogenesis. Biochim Biophys Acta 2011; 1812:141-150.
7. Rahangdale S, Yeh S Y, Malhotra A, Veves A. Therapeutic interventions and oxidative stress in diabetes. Frontiers in bioscience: a journal and virtual library 2009; 14:192-209.
8. Reynolds A, Laurie C, Mosley R L, Gendelman H E. Oxidative stress and the pathogenesis of neurodegenerative disorders. International review of neurobiology 2007; 82:297-325.
9. Kinnula V L, Crapo J D. Superoxide dismutases in the lung and human lung disease. Am J Resp Crit Care Med 2003; 167:1600-1619.
10. Morris A H, Kinnear G, Wan W-Y H, Wyss D, Bahra P, Stevenson C S. Comparison of cigarette smoke-induced acute inflammation in multiple strains of mice and the effect of a matrix metalloproteinase inhibitor on these responses. J Pharmacol Exp Therap 2008; 327:851-862.
11. Taraseviciene-Stewart L, Voelkel N F. Molecular pathogenesis of emphysema. J Clin Invest 2008; 118:394-402.
12. Olanow C W, Jenner P, Youdim M B H. Neurodegeneration and neuroprotection in parkinson's disease. London; San Diego: Academic Press; 1996.
13. Friedman D S, Wolfs R C, O'Colmain B J, Klein B E, Taylor H R, West S, Leske M C, Mitchell P, Congdon N, Kempen J. Prevalence of open-angle glaucoma among adults in the united states. Arch Ophthalmol 2004; 122:532-538.
14. Tezel G. Oxidative stress in glaucomatous neurodegeneration: Mechanisms and consequences. Prog Retin Eye Res 2006; 25:490-513.
15. Tamm E R, Russell P, Johnson D H, Piatigorsky J. Human and monkey trabecular meshwork accumulate alpha b-crystallin in response to heat shock and oxidative stress. Invest Ophthalmol Vis Sci 1996; 37:2402-2413.
16. Mozaffarieh M, Flammer J. Is there more to glaucoma treatment than lowering iop? Surv Ophthalmol 2007; 52 Suppl 2:S174-179.
17. Zanon-Moreno V, Marco-Ventura P, Lleo-Perez A, Pons-Vazquez S, Garcia-Medina J J, Vinuesa-Silva I, Moreno-Nadal M A, Pinazo-Duran M D. Oxidative stress in primary open-angle glaucoma. J Glaucoma 2008; 17:263-268.
18. Aslan M, Dogan S, Kucuksayan E. Oxidative stress and potential applications of free radical scavengers in glaucoma. Redox Rep 2013; 18:76-87.
19. Moreno M C, Campanelli J, Sande P, Sanez D A, Keller Sarmiento M I, Rosenstein R E. Retinal oxidative stress induced by high intraocular pressure. Free Radic Biol Med 2004; 37:803-812.
20. Yildirim O, Ates N A, Ercan B, Muslu N, Unlu A, Tamer L, Atik U, Kanik A. Role of oxidative stress enzymes in open-angle glaucoma. Eye (Lond) 2005; 19:580-583.
21. Pinazo-Duran M D, Zanon-Moreno V, Garcia-Medina J J, Gallego-Pinazo R. Evaluation of presumptive biomarkers of oxidative stress, immune response and apoptosis in primary open-angle glaucoma. Curr Opin Pharmacol 2013; 13:98-107.
22. Schreibelt G, van Horssen J, van Rossum S, Dijkstra C D, Drukarch B, de Vries H E. Therapeutic potential and biological role of endogenous antioxidant enzymes in multiple sclerosis pathology. Brain Res Rev 2007; 56:322-330.
23. Lipinski C A. Drug-like properties and the causes of poor solubility and poor permeability. Journal of pharmacological and toxicological methods 2000; 44:235-249.
24. Pardridge W M. Blood-brain barrier drug targeting: The future of brain drug development. Molecular interventions 2003; 3:90-105, 151.
25. Rahman I. Antioxidant therapeutic advances in copd. Ther Adv Respir Dis 2008; 2:351-374.
26. Estevez A Y, Erlichman J S. Cerium oxide nanoparticles for the treatment of neurological oxidative stress diseases. In: Andreescu S, editor. Oxidative stress: Diagnostics, prevention, and therapy. Washington, D.C.: American Chemical Society; 2011. p. 255-288.
27. Korsvik C, Patil S, Seal S, Self W T. Superoxide dismutase mimetic properties exhibited by vacancy engineered ceria nanoparticles. Chem Commun (Camb) 2007:1056-1058.
28. Andreescu A, Ornatska M, Erlichman J S, Estevez A Y, Leiter J C. Biomedical applications of metal oxide nanoparticles. In: Matijević E, editor. Fine particles in medicine and pharmacy. New York: Springer Science+Business Media, LLC; 2012. p. 57-100.
29. Ganesana M, Erlichman J S, Andreescu S. Real-time monitoring of superoxide accumulation and antioxidant activity in a brain slice model using an electrochemical cytochrome c biosensor. Free Radic Biol Med 2012; 53:2240-2249.
30. Yokel R A, Florence R L, Unrine J M, Tseng M T, Graham U M, Wu P Y K, Grulke E A, Sultana R, Hardas S S, Butterfield D A. Biodistribution and oxidative stress effects of a systematically-introduced commercial ceria engineered nanomaterial. Nanotoxicology 2009; 3:234-248.
31. Yokel R A, Au T C, MacPhail R, Hardas S S, Butterfield D A, Sultana R, Goodman M, Tseng M T, Dan M, Haghnazar H, Unrine J M, Graham U M, Wu P, Grulke E A. Distribution, elimination, and biopersistence to 90 days of a systemically introduced 30 nm ceria-engineered nanomaterial in rats. Toxicol Sci 2012; 127:256-268.
32. Hardas S S, Butterfield D A, Sultana R, Tseng M T, Dan M, Florence R L, Unrine J M, Graham U M, Wu P, Grulke E A, Yokel R A. Brain distribution and toxicological evaluation of a systemically delivered engineered nanoscale ceria. Toxicol Sci 2010; 116:562-576.
33. Heckman K, DeCouteau W, Estevez A Y, Reed K, Constanzo W, Sanford D, Leiter J C, Clauss J, Knapp K, Gomez C, Mullen p, Rathburn E, Prime K, Marini J, Patchevsky J, Patchevsky A S, Hailstone R K, Erlichman J S. Custom cerium oxide nanoparticles protect against a free radical mediated autoimmune degenerative disease in the brain. ACS Nano 2013; 7:10582-10596.
34. Dowding J M, Das S, Kumar A, Dosani T, McCormack R, Gupta A, Sayle T X, Sayle D C, von Kalm L, Seal S, Self W T. Cellular interaction and toxicity depend on physicochemical properties and surface modification of redox-active nanomaterials. ACS Nano 2013; 7:4855-4868.
35. Walkey C D, Olsen J B, Guo H, Emili A, Chan W C. Nanoparticle size and surface chemistry determine serum protein adsorption and macrophage uptake. J Am Chem Soc 2012; 134:2139-2147.
36. Asati A, Santra S, Kaittanis C, Perez J M. Surface-charge-dependent cell localization and cytotoxicity of cerium oxide nanoparticles. ACS Nano 2010; 4:5321-5331.
37. Rzigalinski B A, Danielsen I, Strawn E T, Cohen C A, Liang C. Nanoparticles for cell engineering—a radical concept. In: Kumar CSSR, editor. Tissue, cell and organ engineering. Weinhem: Wiley-VCH Verlag GmbH & Co.; 2006.
38. Schubert D, Dargusch R, Raitano J, Chan S-W. Cerium and yttrium oxide nanoparticles are neuroprotective. Biochem Biophys Res Comm 2006; 342:86-91.
39. Das M, Patil S, Bhargava N, Kang J-F, Riedel L M, Seal S, Hickman J J. Auto-catalytic ceria nanoparticles offer neuroprotection to adult rat spinal cord neurons. Biomaterials 2007; 28:1918-1925.
40. Hirst S M, Karakoti A S, Singh S, Self W, Tyler R D, Seal S, Reilly C M. Bio-distribution and in vivo antioxidant effects of cerium oxide nanoparticles in mice. Environ Toxicol 2011.
41. Kong L, Cai X, Zhou X, Wong L L, Karakoti A S, Seal S, McGinnis J F. Nanoceria extend photoreceptor cell lifespan in tubby mice by modulation of apoptosis/survival signaling pathways. Neurobiol Dis 2011; 42:514-523.
42. Kim C K, Kim T, Choi I Y, Soh M, Kim D, Kim Y J, Jang H, Yang H S, Kim J Y, Park H K, Park S P, Park S, Yu T, Yoon B W, Lee S H, Hyeon T. Ceria nanoparticles that can protect against ischemic stroke. Angew Chem Int Ed Engl 2012.
43. Niu J, Azfer A, Rogers L M, Wang X, Kolattukudy P E. Cardioprotective effects of cerium oxide nanoparticles in a transgenic murine model of cardiomyopathy. Cardiovasc Res 2007; 73:549-559.
44. Bach R D, Dmitrenko O. The effect of substitutents on the strain energies of small ring compounds. The Journal of organic chemistry 2002; 67:2588-2599.
45. Gray P, Williams A. Chemistry of free radicals containing oxygen. Part 3.—thermochemistry and reactivity of the higher alkoxyl radicals ro. Trans Faraday Soc 1959; 55:760-777.
46. Luo Y R. Comprehensive handbook of chemical bond energies. Boca Raton: CRC Press; 2007.
47. Crc handbook of chemistry and physics. Boca Raton: CRC Press; 2010.
48. Trikha S, Jeremic A M. Clustering and internalization of toxic amylin oligomers in pancreatic cells require plasma membrane cholesterol. J Biol Chem 2011; 286:36086-36097.
49. Janciauskiene S, Ahren B. Fibrillar islet amyloid polypeptide differentially affects oxidative mechanisms and lipoprotein uptake in correlation with cytotoxicity in two insulin-producing cell lines. Biochemical and biophysical research communications 2000; 267:619-625.
50. Clark A, Nilsson M R. Islet amyloid: A complication of islet dysfunction or an aetiological factor in type 2 diabetes? Diabetologia 2004; 47:157-169.
51. Chanteau B, Fresnais J, Berret J F. Electrosteric enhanced stability of functional sub-10 nm cerium and iron oxide particles in cell culture medium. Langmuir 2009; 25:9064-9070.
52. Nunez N O, Liviano S R, Ocana M. Citrate mediated synthesis of uniform monazite lnpo4 (ln=la, ce) and ln:Lapo4 (ln=eu, ce, ce+tb) spheres and their photoluminescence. Journal of colloid and interface science 2010; 349:484-491.
53. Safi S, H., Sandre O, Mignet N, Berret J-F. Interactions between sub-10-nm iron and cerium oxide nanoparticles and 3t3 fibroblasts: The role of the coating and aggregation state. Nanotechnology 2010; 21:145103.
54. Tawfik D S. Amidation of carboxyl groups. In: Walker J M, editor. The protein protocols handbook. New York: Springer; 1996. p. 361-362.
55. Fischer M J. Amine coupling through edc/nhs: A practical approach. In: Fischer MJMNJ, Fischer M J, editors. Surface plasmon resonance: Methods and protocols. New York: Springer; 2010. p. 55-73.
56. Hermanson G T. Bioconjugate techniques. Academic Press; 1996.
57. Klibanov A L, Maruyama K, Torchilin V P, Huang L. Amphipathic polyethyleneglycols effectively prolong the circulation time of liposomes. FEBS letters 1990; 268:235-237.
58. Romberg B, Hennink W E, Storm G. Sheddable coatings for long-circulating nanoparticles. Pharm Res 2008; 25:55-71.
59. Dubertret B, Skourides P, Norris D J, Noireaux V, Brivanlou A H, Libchaber A. In vivo imaging of quantum dots encapsulated in phospholipid micelles. Science 2002; 298:1759-1762.
60. Jewett S A, Yoder J A, Ivanisevic A. Surface modifications on in as decrease indium and arsenic leaching under physiolgical conditions. Appl Surf Sci 2012; 261:842-850.
61. Cheraghipour E, Tamaddon A M, Javadpour S, Bruce I J. Peg conjugated citrate-capped magnetite nanoparticles for biomedical applications. J Magn Magn Mater 2013; 328:91-95.
62. Johnsson M, Hansson P, Edwards K. Spherical micelles and other self-assembled structures in dilute aqueous mixtures of poly(ethylene glycol) lipids. J Phys Chem B 2001; 105:8420-8430.
63. Karakoti A S, Das S, Thevuthasan S, Seal S. Pegylated inorganic nanoparticles. Angew Chem Int Ed Engl 2011; 50:1980-1994.
64. Counihan T, Penney J. Regional dopamine transporter gene expression in the substantia nigra from control and parkinson's disease brains. J Neurol Neurosurg Psychiatry 1998; 65:164-169.
65. Baillet A, Chanteperdrix V, Trocme C, Casez P, Garrel C, Besson G. The role of oxidative stress in amyotrophic lateral sclerosis and parkinson's disease. Neurochemical research 2010; 35:1530-1537.
66. Poewe W, Mahlknecht P, Jankovic J. Emerging therapies for parkinson's disease. Current opinion in neurology 2012; 25:448-459.
67. Han G C, Liu Y N. Synthesis, characterization and fluorescent properties of cerium(iii) glutathione complex. Luminescence: the journal of biological and chemical luminescence 2010; 25:389-393.
68. Pardridge W M. The blood-brain barrier: Bottleneck in brain drug development. NeuroRx: the journal of the American Society for Experimental Neuro Therapeutics 2005; 2:3-14.
69. Hendriks J J, Alblas J, van der Pol S M, van Tol E A, Dijkstra C D, de Vries H E. Flavonoids influence monocytic gtpase activity and are protective in experimental allergic encephalitis. J Exp Med 2004; 200:1667-1672.
70. Stanislaus R, Gilg A G, Singh A K, Singh I. N-acetyl-l-cysteine ameliorates the inflammatory disease process in experimental autoimmune encephalomyelitis in lewis rats. J Autoimmune Dis 2005; 2:4.
71. Moriya M, Nakatsuji Y, Miyamoto K, Okuno T, Kinoshita M, Kumanogoh A, Kusunoki S, Sakoda S. Edaravone, a free radical scavenger, ameliorates experimental autoimmune encephalomyelitis. Neurosci Lett 2008; 440:323-326.
72. Marracci G H, Jones R E, McKeon G P, Bourdette D N. Alpha lipoic acid inhibits t cell migration into the spinal cord and suppresses and treats experimental autoimmune encephalomyelitis. J Neuroimmunol 2002; 131:104-114.
73. Aktas O, Waiczies S, Smorodchenko A, Dorr J, Seeger B, Prozorovski T, Sallach S, Endres M, Brocke S, Nitsch R, Zipp F. Treatment of relapsing paralysis in experimental encephalomyelitis by targeting thl cells through atorvastatin. J Exp Med 2003; 197:725-733.

What is claimed is:

1. A multi-layered encapsulated cerium oxide nanoparticle (“CeNP”) comprising a cerium oxide nanoparticle and a ligand shell, wherein the CeNP can optionally comprise a hydrocarbon addition, an electron shuttling system, a lipid shell, a targeting molecule attachment, or combinations thereof.

2. The CeNP of claim 1, wherein the ligand shell is the inner most linkage to the CeNP.

3. The CeNP of claim 1, wherein the ligand shell comprises a hydrocarbon having 2 to 40 carbons in length.

4. The CeNP of claim 3, wherein the ligand shell comprises chelating carboxylic acids.

5. The CeNP of claim 4, wherein the chelating carboxylic acids comprise at least one of butyl, t-butyl, hexyl, decyl, hexyldecyl carboxylic acids, or hydrocarbons with opposing functionalities of carboxylic acids and ethers, esters, epoxides, peroxides, thiols or acetals or combinations thereof.

6. The CeNP of claim 1, wherein the ligand shell comprises stearic acid, oleic acid, polyacrylate, citric acid, or combinations thereof.

7. The CeNP of claim 1, wherein the hydrocarbon addition comprises n-terminal amine hydrocarbons.

8. The CeNP of claim 7, wherein the hydrocarbon addition is a linker.

9. The CeNP of claim 7, wherein the amine hydrocarbons comprise butyl, t-butyl, hexyl, decyl, hexyldecyl amines or amines with dual functionalities such as ω-terminal ethers, esters, epoxide, peroxides, thiols, acetals.

10. The CeNP of claim 1, wherein the electron shuttling system comprises large conjugated systems or a system of fixed benzyl rings or alternating double bonds on a hydrocarbon chain.

11. The CeNP of claim 1, wherein the lipid shell comprises long chain, large lipids.

12. The CeNP of claim 1, wherein the lipid shell comprises phospholipids, sphingolipids or sterols with various headgroup and tail options.

13. The CeNP of claim 1, wherein the targeting molecule attachment comprises small molecules that couple using carboxylic acid, thiol groups or amines.

14. The CeNP of claim 1, wherein the targeting molecule attachment comprises L-DOPA, dopamine, serotonin, acetylcholine, 6OHDA, derivatives thereof, or peptides.

15. A method of controlling and directing CeNP action against reactive oxygen species, the method comprising:
making the CeNP with at least one unzipping formation;
exposing the CeNP to the presence of the reactive oxygen species or free radicals, whereby the anti-oxidant activity of the CeNP is made available to sites where the reactive oxygen species or free radicals are formed or abundant.

16. The method of claim 15, wherein the CeNP comprises a lipid encapsulation linked to a treated surface of the cerium.

17. The method of claim 16, wherein the CeNP comprises short linking hydrocarbons that facilitate the formation of a lipid coat on the modified surface of the cerium.

18. The method of claim 17, wherein embedded chemical bonds for both or either a ligand shell and lipid shell are susceptible to attack by the reactive oxygen species or free radicals.

19. A method for limiting interactions of a CeNP with blood and tissue in the body, the method comprising:
administering a multi-layered, encapsulated cerium oxide nanoparticle (“CeNP”) to a subject in need thereof, wherein the CeNP is formed to limit intrinsic anti-oxidative activities of the CeNP.

20. The method of claim 19, wherein the CeNP is passivated with carbon chains or other bulky additions such as tert-butyl, cycloalkanes, dendritic structures, polypropylene functionalities.
